(19) United States
(12) Patent Application Publication
(10) Pub. No.: US 2023/0167545 A1
(43) Pub. Date: Jun. 1, 2023

(54) STABILIZATION OF CARBON DEPOSITION PRECURSORS LIKE C2H2

(71) Applicant: Lam Research Corporation, Fremont, CA (US)

(72) Inventors: Kapu Sirish Reddy, Portland, OR (US); Adrien LaVoie, Newberg, OR (US)

(21) Appl. No.: 17/905,955

(22) PCT Filed: Mar. 12, 2021
(86) PCT No.: PCT/US2021/022162

§ 371 Date: Sep. 9, 2022

Related U.S. Application Data
(60) Provisional application No. 62/989,382, filed on Mar. 13, 2020.

(51) Int. Cl. C23C 16/26

(52) U.S. Cl. CPC C23C 16/26

(57) ABSTRACT

The present disclosure relates to compositions including a mixture or solution of acetylene and a stabilizer. In particular embodiments, the composition is a stabilized composition including pressurized acetylene.

### RELATED APPLICATIONS

A PCT Request Form is filed concurrently with this specification as part of the present application. Each application that the present application claims benefit of or priority to as identified in the concurrently filed PCT Request Form is incorporated by reference herein in its entirety and for all purposes.

### FIELD

The present disclosure relates to compositions including a mixture or solution of acetylene and a stabilizer. In particular embodiments, the composition is a stabilized composition including pressurized acetylene.

### BACKGROUND

Acetylene is known to be explosive when pressurized above 15 psig (pounds per square inch gauge). To avoid explosions during storage and transportation, acetylene may be stored in a canister or cylinder filled with a porous material having a stabilizer. Acetone is commonly used as a stabilizer, in part because acetylene is highly soluble in acetone. One volume of liquid acetone can absorb twenty-five volumes of gaseous acetylene at temperatures of about 15° C. under atmospheric pressure and will continue to absorb an additional twenty-five volumes of acetylene for every additional atmosphere of pressure to which acetylene is subjected. In using acetylene as a precursor for the deposition of carbon films in chemical vapor deposition (CVD) processes, the cylinders are connected to a gas line. Acetylene is then fed into the line for introduction to the deposition chamber.

Background and contextual descriptions contained herein are provided solely for the purpose of generally presenting the context of the disclosure. Much of this disclosure presents work of the inventors, and simply because such work is described in the background section or presented as context elsewhere herein does not mean that it is admitted to be prior art.

### SUMMARY

The present invention relates, in part, to a composition including acetylene and one or more stabilizers.

Accordingly, in a first aspect, the composition includes a mixture or solution of: acetylene; and a stabilizer including a ketone having a vapor pressure of about 30 Torr or lower at 25° C. In particular embodiments, the acetylene and stabilizer are stored in a containment vessel.

In a second aspect, the composition includes a mixture or solution of: acetylene; and a stabilizer including a ketone. In some embodiments, the ketone is selected from acetylacetone (acac), 2-butanone, 2-pentanone, 2-hexanone, 2-heptanone, 2-octanone, 2-nonanone, 2-decanone, 3-pentanone, 3-heptanone, 3-octanone, 3-nonanone, 3-decanone, and aromatic aldehydes such as acetophenone, 3-hydroxyacetophenone, cyclohexanone, benzophenone, butyrophenone, acetylpyrazine, 2-acetyl pyridine, acrylophenone, capillin, dibenzoylmethane, indenone, 1-indanone, paroxypropione, phenylglyoxal, piceol, propriophenone, pyridoxal, 2,4,6-trihydroxyacetophenone, 2,4,5-trihydroxyacetophenone, and valerophenone, as well as mixtures thereof. In particular embodiments, the acetylene and stabilizer are stored in a containment vessel.

In a third aspect, the composition includes a mixture or solution of: acetylene; and a stabilizer including an ionic liquid (e.g., any described herein, such as an ionic liquid including one or more cationic moieties and one or more anionic moieties). Exemplary, non-limiting ionic liquids include a cation selected from imidazolium, pyridinium, ammonium, phosphonium, thiazolium, and triazolium. In particular embodiments, the acetylene and stabilizer are stored in a containment vessel.

In a fourth aspect, the composition includes a mixture or solution of: acetylene; and a stabilizer selected from carbenes and silylenes. In one embodiment, the stabilizer is a carbene selected from transition metal carbene complexes, N-heterocyclic carbenes, and methylenes. In other embodiments, the stabilizer is a silylene (e.g., an N-heterocyclic silylene). In particular embodiments, the acetylene and stabilizer are stored in a containment vessel.

In a fifth aspect, the composition includes a mixture or solution of: acetylene; and a stabilizer including an aldehyde (e.g., any described herein). In some embodiments, the stabilizer is benzaldehyde. In particular embodiments, the acetylene and stabilizer are stored in a containment vessel.

In a sixth aspect, the composition includes a mixture or solution of: acetylene; and a stabilizer including an ester having a vapor pressure of about 90 Torr or lower at 25° C. In particular embodiments, the acetylene and stabilizer are stored in a containment vessel.

In a seventh aspect, the composition includes a mixture or solution of: acetylene; and a stabilizer including an amide having a vapor pressure of about 3 Torr or lower at 25° C. In particular embodiments, the acetylene and stabilizer are stored in a containment vessel.

In an eighth aspect, the composition includes a mixture or solution of: acetylene; and a stabilizer including an ether (e.g., furan, tetrahydrofuran, and/or pyran). In particular embodiments, the acetylene and stabilizer are stored in a containment vessel.

In a ninth aspect, the composition includes a mixture or solution of: acetylene; and a stabilizer including an amine (e.g., any described herein). In some embodiments, the amine selected from N-ethyldiisopropylamine, trimethylamine, dimethylamine, methylamine, triethylamine, and tert-butylamine. In other embodiments, the amine is an aromatic amine (e.g., aniline, N,N-dimethylaniline, piperidine, pyrrole, pyrrolidine, pyridine, piperidine, imidazole, or pyrimidine). In particular embodiments, the acetylene and stabilizer are stored in a containment vessel.

In a tenth aspect, the composition includes a mixture or solution of: acetylene; and a stabilizer including an imine (e.g., any described herein). In some embodiments, the imine is selected from Schiff bases and 2,5-cyclohexadiene-1,4-diimine. In particular embodiments, the acetylene and stabilizer are stored in a containment vessel.

In an eleventh aspect, the composition includes a mixture or solution of: acetylene; and a stabilizer including a nitrile having a vapor pressure of about 80 Torr or lower at 25° C. In particular embodiments, the acetylene and stabilizer are stored in a containment vessel.

In a twelfth aspect, the composition includes a mixture or solution of: acetylene; and a stabilizer including a nitrogen-containing saturated heterocyclic ring compound. In some embodiments, the nitrogen-containing saturated heterocyclic ring compound is selected from pyrrolidine and morpholine. In particular embodiments, the acetylene and stabilizer are stored in a containment vessel.

In a thirteenth aspect, the composition includes a mixture or solution of: acetylene; and a stabilizer including a nitrogen-containing unsaturated heterocyclic ring compound. In some embodiments, the nitrogen-containing unsaturated heterocyclic ring compound is selected from pyridine, pyrazine, imidazole, pyrrole, N-iminopyridinium ylide, triazole, thiazole, and substituted derivatives of any of these, such as N-methylimidizole, 2,6-lutidine, and 4-N,N-dimethylaminopyridine. In particular embodiments, the acetylene and stabilizer are stored in a containment vessel.

In a fourteenth aspect, the composition includes a mixture or solution of: acetylene; and a stabilizer including a mixed electron donor compound including a pi bond and an atom having a lone electron pair. In some embodiments, the mixed electron donor compound is selected from acetone, imine, 2-methyl-2-butenone, triazole, and thiazole. In particular embodiments, the acetylene and stabilizer are stored in a containment vessel.

In a fifteenth aspect, the composition includes a mixture or solution of: acetylene; and a stabilizer including a phosphorus-containing compound, in which the phosphorus atom has a lone electron pair. In some embodiments, the phosphorus-containing compound is selected triphenylphosphine and triphenylphosphine oxide. In particular embodiments, the acetylene and stabilizer are stored in a containment vessel.

In a sixteenth aspect, the composition includes a mixture or solution of: acetylene; and a stabilizer including a sulfur-containing compound, in which the sulfur atom has a lone electron pair. In some embodiments, the sulfur-containing compound is selected of thiophene, thiazolium, thiazole, 2-methylthiophene, 3-methylthiophene, 2,4-dimethylthiophene, benzothiophene, and 2-methylbenzothiophene. In particular embodiments, the acetylene and stabilizer are stored in a containment vessel.

In a seventeenth aspect, the composition includes a mixture or solution of: acetylene; and a stabilizer including an unsaturated linear or branched hydrocarbon. In some embodiments, the unsaturated linear or branched hydrocarbon is selected from butene, butadiene, 1-butyne, propyne, pentene, octene, heptene, hexyne, 1-heptyne, 1-octyne, 1-nonyne, and 1-decyne. In particular embodiments, the acetylene and stabilizer are stored in a containment vessel.

In an eighteenth aspect, the composition includes a mixture or solution of: acetylene; and a stabilizer that is an unsaturated ring hydrocarbon having a vapor pressure of about 5 Torr or lower at 25° C. In particular embodiments, the acetylene and stabilizer are stored in a containment vessel.

In a nineteenth aspect, the composition includes a mixture or solution of: acetylene; and a stabilizer including a non-aromatic unsaturated ring hydrocarbon. In some embodiments, the stabilizer is cyclopentene or cyclohexene. In particular embodiments, the acetylene and stabilizer are stored in a containment vessel.

In a twentieth aspect, the composition (e.g., the stabilized composition) includes an acetylene (e.g., a pressurized acetylene); and a stabilizer including a nitrogen atom, wherein the stabilizer is not dimethylformamide, not dimethylacetamide, not N-methyl-2-pyrrolidone, and not acetonitrile. In some embodiments, the stabilizer further includes optionally substituted heterocyclyl. In other embodiments, the stabilizer is an amide (e.g., a dialkyl amide, a pyrrolidone, an acetamide, a morpholide, an ester amide, and a cyclic amide), an amine (e.g., an amine including optionally substituted aliphatic, optionally substituted alkyl, optionally substituted aryl, optionally substituted aliphatic-aryl, optionally substituted alkyl-aryl, optionally substituted alkenyl-aryl, optionally substituted alkynyl-aryl, optionally substituted heteroaliphatic, optionally substituted heteroalkyl, optionally substituted heterocyclyl, or optionally substituted alkyl-heterocyclyl), a guanidine, an imine, or an N-heterocyclic carbene. In yet other embodiments, the stabilizer is a Schiff base.

In a twenty-first aspect, the composition (e.g., the stabilized composition) includes an acetylene (e.g., a pressurized acetylene); and a stabilizer that is a heterocycle, wherein the stabilizer is not 1,3-dioxolane, not 1,4-dioxane, and not N-methyl-2-pyrrolidone. In some embodiments, the heterocycle is an aromatic heterocycle, a bicyclic heterocycle, a cyclic ether, a cyclic ester, a cyclic carbonate ester, a cyclic amine, a cyclic amide, or an N-heterocyclic carbene. In other embodiments, the heterocycle includes one or more heteroatoms selected from the group consisting of nitrogen (N), oxygen (O), or sulfur (S).

In a twenty-second aspect, the composition (e.g., the stabilized composition) includes an acetylene (e.g., a pressurized acetylene); and a stabilizer that is a substituted aromatic hydrocarbon having one or more substitutions, wherein the one or more substitutions are selected from the group consisting of halo, amine, and optionally substituted C₂₋₈ alkyl. In some embodiments, the aromatic hydrocarbon includes a first substitution selected from the group consisting of halo, amine, and optionally substituted C₂₋₈ alkyl and a second substitution selected from the group consisting of halo, amine, and optionally substituted alkyl.

In a twenty-third aspect, the composition (e.g., the stabilized composition) includes an acetylene (e.g., a pressurized acetylene); and a stabilizer that is an optionally substituted alkene or an optionally substituted alkyne. In some embodiments, the optionally substituted alkene is a diene.

In a twenty-fourth aspect, the composition (e.g., the stabilized composition) includes an acetylene (e.g., a pressurized acetylene); and a stabilizer that is an aldehyde or an ether, wherein the stabilizer is not 1,3-dioxolane and not 1,4-dioxane. In some embodiments, the aldehyde includes an optionally substituted aliphatic, optionally substituted alkyl, optionally substituted aryl, optionally substituted aliphatic-aryl, optionally substituted alkyl-aryl, optionally substituted alkenyl-aryl, optionally substituted alkynyl-aryl, optionally substituted heteroaliphatic, optionally substituted heteroalkyl, optionally substituted heterocyclyl, or optionally substituted alkyl-heterocyclyl having one or more aldehyde moieties. In other embodiments, the ether includes optionally substituted aliphatic, optionally substituted alkyl, optionally substituted aryl, optionally substituted aliphatic-aryl, optionally substituted alkyl-aryl, optionally substituted alkenyl-aryl, optionally substituted alkynyl-aryl, optionally substituted heteroaliphatic, optionally substituted heteroalkyl, optionally substituted heterocyclyl, or optionally substituted alkyl-heterocyclyl.

In a twenty-fifth aspect, the composition (e.g., the stabilized composition) includes an acetylene (e.g., a pressurized acetylene); and a stabilizer that is an ester selected from the group consisting of a cyclic ester, a glycol based ester, a lactate, a carbonate ester, an amino ester, and a diester. In some embodiments, the ester includes one or more amino.

In a twenty-sixth aspect, the composition (e.g., the stabilized composition) includes an acetylene (e.g., a pressurized acetylene); and a stabilizer that is a cyclic ketone, an aryl ketone, a dione, or a trione. In some embodiments, the stabilizer includes optionally substituted cycloalkyl or optionally substituted aryl.

In a twenty-seventh aspect, the composition (e.g., the stabilized composition) includes an acetylene (e.g., a pressurized acetylene); and a stabilizer that is a carbene or a carbene derivative. In some embodiments, the carbene includes optionally substituted aliphatic, optionally substituted alkyl, optionally substituted aryl, optionally substituted aliphatic-aryl, optionally substituted alkyl-aryl, optionally substituted alkenyl-aryl, optionally substituted alkynyl-aryl, or optionally substituted heterocyclyl. In other embodiments, the carbene or the carbene derivative includes a thiazol-2-ylidene moiety, a dihydroimidazol-2-ylidene moiety, an imidazol-2-ylidene moiety, a phosphinocarbene moiety, a triazol-5-ylidene moiety, or a cyclopropenylidene moiety. In yet other embodiments, the carbene or the carbene derivative is selected from an aminothiocarbene compound, an aminooxycarbene compound, a diaminocarbene compound, a heteroamino carbene compound, a 1,3-dithiolium carbene compound, a mesoionic carbene compound, a cyclic alkyl amino carbene compound, a boranylidene compound, a silylene compound, a stannylene compound, a nitrene compound, a phosphinidene compound, and a foiled carbene compound.

In a twenty-eighth aspect, the composition (e.g., the stabilized composition) includes an acetylene (e.g., a pressurized acetylene); and a stabilizer that is a metal compound, an onium compound, an organosulfur compound, or an organophosphorus compound, wherein the stabilizer is not dimethylsulfoxide. In some embodiments, the onium compound is selected from a nitronium ion, a nitrosonium ion, a bis(triphenylphosphine) iminium ion, an iminium ion, a diazenium ion, a guanidinium ion, a nitrilium ion, a diazonium ion, a pyridinium ion, a pyrylium ion, and a thionitrosyl ion. In other embodiments, the organosulfur compound is selected from a thioester, a sulfoxide, a sulfone, a thiosulfinate, a sulfimide, a sulfoximide, a sulfonediimine, an S-nitrosothiol, a thioketone, a thioaldehyde, a thioamide, a sulfonium, an oxosulfonium, and a thiocarbonyl ylide. In yet other embodiments, the organophosphorus compound is selected from a phosphate ester, a phosphate amide, a phosphonate, a phosphinate, a phosphine oxide, a phosphine imide, and a phosphonium salt.

In a twenty-ninth aspect, the composition (e.g., the stabilized composition) includes an acetylene (e.g., a pressurized acetylene); and a stabilizer that is an ionic liquid. In some embodiments, the ionic liquid includes a cationic moiety including imidazolium, pyridinium, pyrrolidinium, ammonium, phosphonium, thiazolium, or triazolium; and further includes an anionic moiety including tetrafluoroborate, hexafluorophosphate, bistriflimide, triflate, acetate, trifluoroacetate, triflimide, halide, bis(trifluoromethylsulfonyl)imide, methylsulfate, ethyl sulfate, docusate, or dicyanamide.

In any embodiment herein, the acetylene and the stabilizer are stored in a containment vessel.

In any embodiment herein, the acetylene and the stabilizer are stored under a pressure of at least about 200 psi.

These and other features of the disclosure will be presented in more detail below with reference to the associated drawings.

### BRIEF DESCRIPTION OF THE DRAWINGS

FIG. 1 depicts a non-limiting process flow diagram for forming an ashable hardmask.

FIG. 2 depicts a non-limiting block diagram for a deposition system.

FIG. 3 depicts a non-limiting process flow diagram for treating an acetylene gas stream.

FIG. 4 depicts a non-limiting block diagram for a pre-processing module.

FIG. 5 depicts a non-limiting schematic diagram for a trap in a pre-processing module.

FIG. 6 depicts a non-limiting block diagram for a reactor.

### DETAILED DESCRIPTION

### Acetylene for Deposition Processes in IC Fabrication

Acetylene is a precursor for carbon deposition. Due to its low ionization energy, it can perform well for carbon deposition through plasma enhanced chemical vapor deposition (PECVD). One type of carbon film deposited using acetylene is the ashable hard mask (AHM).

However, acetylene may be used in depositing various other types of films, and it is not limited to the deposition of ashable hardmask films. This disclosure pertains to any carbon-based film using acetylene as a precursor in semiconductor processing.

Acetylene for semiconductor processing may be supplied in cylinders (also referred to as bottles) storing 200-500 cubic feet of acetylene (at standard temperature and pressure), where acetylene is dissolved in acetone. Acetylene vendors include Dow Chemicals, Air Products, PraxAir, Air Gas, Linde Gas, and other suppliers.

As mentioned, acetylene bottles used in semiconductor processing are sometimes stabilized with acetone. Acetone is less toxic and less expensive than some other stabilizers. However, it would be useful to have other stabilizers.

### Examples of Stabilizers

As indicated, acetylene storage requires a stabilizer. Currently, industry stabilizers include acetone and dimethylformamide (DMF). Additional acetylene stabilizers are described herein.

In certain embodiments, an acetylene stabilizer has a vapor pressure of about 7000 Torr or lower at 25° C. In certain embodiments, an acetylene stabilizer has a vapor pressure of about 700 Torr or lower at 25° C. In certain embodiments, an acetylene stabilizer has a vapor pressure of about 150 Torr or lower at 25° C. In certain embodiments, an acetylene stabilizer has a vapor pressure of about 75 Torr or lower at 25° C. In certain embodiments, an acetylene stabilizer has a vapor pressure of about 10 Torr or lower at 25° C.

In certain embodiments, the stabilizer is a nucleophile, such as a compound having a lone pair of electrons on an atom and/or a covalent pi bond. In some cases, the stabilizer has more than one lone pairs of electrons, more than one pi bond, or a combination of one or more lone pairs of electrons and one or more pi bonds.

To be clear, compounds having a “lone pair” of electrons have an orbital with two electrons that do not bond with any other atoms. In other words, the lone pair electrons are not shared with any other atoms.

Pi bonds and lone pair electrons can stabilize acetylene by a stacking mechanism, in which pi bonds from acetylene's carbon-carbon triple bond stably interact with a pi bond or an orbital from a lone pair of electrons. A similar stabilization mechanism is “pi stacking” that provides attractive, non-covalent interactions between aromatic groups.

One class of acetylene stabilizer for use with this disclosure is the ionic liquids. In certain embodiments, the ionic liquids have ions with a heterocyclic ring, which may be aromatic. Examples of ionic liquid acetylene stabilizers include imidazolium, pyridinium, ammonium, phosphonium, thiazolium, and triazolium cations. Examples of anions include tetrafluoroborate (BF⁴⁻), hexafluorophosphate (PF⁶⁻), triflate (CF₃SO₃⁻), triflimide, dicyanamide (C₂N₃⁻), and the like. Specific examples include cation-anion pairs of any of these. In certain embodiments, the ionic liquid stabilizer has a vapor pressure of about 20 Torr or lower at 25° C.

Another class of acetylene stabilizer for use with this disclosure is the carbenes. Carbenes have general formula R¹R²C: (where “:” is an unbonded, lone electron pair). One class of stable carbenes that may be used to stabilize acetylene are the transition metal carbene complexes. These include, e.g., Schrock or Fisher carbenes of general formula M=R having the classic structures illustrated in Scheme 1 (below).

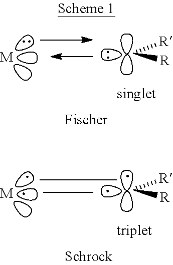

Examples of other carbene-type acetylene stabilizers include N-heterocyclic carbenes, methylenes, and silicon derivatives including silylenes that may be, for example, N-heterocyclic silylenes (persistent silylenes) or of the type R¹R²—Si, where: is a lone electron pair. In certain embodiments, the carbene or silylene stabilizer has a vapor pressure of about 7000 Torr or lower at 25° C. In certain embodiments, the carbene stabilizer has a vapor pressure of about 700 Torr or lower at 25° C.

Another class of acetylene stabilizer for use with this disclosure is the organic, oxygen-containing compounds, in which the oxygen atom has a lone electron pair. One example of an oxygen-containing moiety for this class of stabilizer is the carbonyl group. Another example of such moiety is the ether group. Another example of such moiety is the alcohol group. Examples of organic carbonyl compounds include ketones, aldehydes, carboxylic acids, esters, and amides.

Suitable acetylene stabilizing ketones may have the general formula XYC═O. In certain embodiments, X is the same as Y. In certain embodiments, X and Y are not the same. In certain embodiments, each X and/or Y is independently selected from hydrogen, aliphatic, aryl, heteroaliphatic, or heteroaryl. Exemplary alkyl group includes, but are not limited to, methyl, ethyl, propyl, butyl, isopropyl, and t-butyl. In certain embodiments, aryl rings of the aryl ketones are substituted at the 1, 2, and/or 3 positions; and the aryl rings can be substituted with one or more functional groups selected from aliphatic, alkoxy, amide, amine, thioether, haloalkyl, nitro, halo, silyl, cycloaliphatic, aryl, and the like.

Examples of ketones that may serve as acetylene stabilizers include acetylacetone (acac), 2-butanone, 2-pentanone, 2-hexanone, 2-heptanone, 2-octanone, 2-nonanone, 2-decanone, 3-pentanone, 3-heptanone, 3-octanone, 3-nonanone, 3-decanone, and aromatic aldehydes such as acetophenone, 3-hydroxyacetophenone, cyclohexanone, benzophenone, butyrophenone, acetylpyrazine, 2-acetyl pyridine, acrylophenone, capillin, dibenzoylmethane, indenone, 1-indanone, paroxypropione, phenylglyoxal, piceol, propriophenone, pyridoxal, 2,4,6-trihydroxyacetophenone, 2,4,5-trihydroxyacetophenone, and valerophenone. In certain embodiments, the ketone stabilizer has a vapor pressure of about 700 Torr or lower at 25° C. In certain embodiments, the ketone stabilizer has a vapor pressure of about 30 Torr or lower at 25° C.

An example aldehyde that may serve as acetylene stabilizers is benzaldehyde. In certain embodiments, the aldehyde stabilizer has a vapor pressure of about 700 Torr or lower at 25° C. In certain embodiments, the aldehyde stabilizer has a vapor pressure of about 200 Torr or lower at 25° C.

Carboxylic acids may serve as acetylene stabilizers. In certain embodiments, the carboxylic acid stabilizer has a vapor pressure of about 700 Torr or lower at 25° C. In certain embodiments, the carboxylic acid stabilizer has a vapor pressure of about 15 Torr or lower at 25° C.

Esters may serve as acetylene stabilizers. In certain embodiments, the ester stabilizer has a vapor pressure of about 700 Torr or lower at 25° C. In certain embodiments, the ester stabilizer has a vapor pressure of about 90 Torr or lower at 25° C.

Various amides may serve as acetylene stabilizers. In certain embodiments, the amide stabilizer has a vapor pressure of about 700 Torr or lower at 25° C. In certain embodiments, the amide stabilizer has a vapor pressure of about 3 Torr or lower at 25° C.

Examples of ethers that may serve as acetylene stabilizers include furan, tetrahydrofuran, and pyran. In certain embodiments, the ether stabilizer has a vapor pressure of about 700 Torr or lower at 25° C. In certain embodiments, the ether stabilizer has a vapor pressure of about 100 Torr or lower at 25° C.

Examples of alcohols that may serve as acetylene stabilizers include the multifunctional alcohols, such as glycerol or glycerine. In certain embodiments, the alcohol stabilizer has a vapor pressure of about 700 Torr or lower at 25° C. In certain embodiments, the alcohol stabilizer has a vapor pressure of about 100 Torr or lower at 25° C.

Another class of acetylene stabilizer for use with this disclosure is the organic, nitrogen-containing compounds, in which the nitrogen atom has a lone electron pair. Examples of types of compounds in this class include amines, imines, nitriles, nitrogen-containing saturated heterocyclic ring compounds, nitrogen-containing unsaturated heterocyclic ring compounds, and amides.

Examples of amines that may serve as acetylene stabilizers include N-ethyldiisopropylamine, trimethylamine, dimethylamine, methylamine, triethylamine, tert-butylamine, aromatic amines, and heterocyclic amines. Examples of aromatic amines include aniline and its derivatives such as N,N-dimethylaniline, piperidine, pyrrole, pyrrolidine, pyridine, piperidine, imidazole, and pyrimidine. In certain embodiments, the amine stabilizer has a vapor pressure of about 700 Torr or lower at 25° C. In certain embodiments, the amine stabilizer has a vapor pressure of about 100 Torr or lower at 25° C.

Examples of imines that may serve as acetylene stabilizers include Schiff bases and diimines such as 2,5-cyclohexadiene-1,4-diimine. In certain embodiments, the imine stabilizer has a vapor pressure of about 700 Torr or lower at 25° C. In certain embodiments, the imine stabilizer has a vapor pressure of about 100 Torr or lower at 25° C.

An example of a nitrile that may serve as acetylene stabilizers is benzonitrile. In certain embodiments, the nitrile stabilizer has a vapor pressure of about 700 Torr or lower at 25° C. In certain embodiments, the nitrile stabilizer has a vapor pressure of about 80 Torr or lower at 25° C.

Examples of nitrogen-containing saturated heterocyclic ring compounds that may serve as acetylene stabilizers include pyrrolidine, morpholine, and substituted derivatives of any of these. In certain embodiments, the nitrogen-containing saturated heterocyclic ring compound has a vapor pressure of about 700 Torr or lower at 25° C. In certain embodiments, the nitrogen-containing saturated heterocyclic ring compound has a vapor pressure of about 100 Torr or lower at 25° C.

Examples of nitrogen-containing unsaturated heterocyclic ring compounds, including aromatic heterocycles, that may serve as acetylene stabilizers include pyridine, pyrazine, imidazole, pyrrole, N-iminopyridinium ylide, triazole, thiazole, and substituted derivatives of any of these, such as N-methylimidizole, 2,6-lutidine, and 4-N,N-dimethylaminopyridine. In certain embodiments, the nitrogen-containing unsaturated heterocyclic ring compound has a vapor pressure of about 700 Torr or lower at 25° C. In certain embodiments, the nitrogen-containing unsaturated heterocyclic ring compound has a vapor pressure of about 100 Torr or lower at 25° C.

Another class of acetylene stabilizer for use with this disclosure includes mixed electron donor compounds. These are compounds that have at least two distinct electron donors. In certain embodiments, a mixed donor compound has two different types of electron donor (e.g., a pi bond and a lone electron pair). Examples of compounds in this class include acetone imine, 2-methyl-2-butenone, triazole, morpholine, and thiazole. In certain embodiments, the mixed electron donor compound has a vapor pressure of about 700 Torr or lower at 25° C. In certain embodiments, the mixed electron donor compound has a vapor pressure of about 100 Torr or lower at 25° C.

Another class of acetylene stabilizer for use with this disclosure is the phosphorus-containing compounds, in which the phosphorus atom has a lone electron pair. Examples of types of compounds in this class include various phosphines. Examples of phosphine compounds that may be used as stabilizers include triphenylphosphine and triphenylphosphine oxide. In certain embodiments, the phosphorus-containing compound has a vapor pressure of about 700 Torr or lower at 25° C. In certain embodiments, the phosphorus-containing compound has a vapor pressure of about 100 Torr or lower at 25° C.

Another class of acetylene stabilizer for use with this disclosure is the sulfur-containing compounds, in which the sulfur atom has alone electron pair. Examples of types of compounds in this class include aromatic heterocyclic ring compounds such as thiophene, thiazolium, thiazole, and their salts and derivatives, such as 2-methylthiophene, 3-methylthiophene, 2,4-dimethylthiophene, benzothiophene, and 2-methylbenzothiophene. In certain embodiments, the sulfur-containing compounds has a vapor pressure of about 700 Torr or lower at 25° C. In certain embodiments, the sulfur-containing compounds has a vapor pressure of about 100 Torr or lower at 25° C.

Another class of acetylene stabilizer for use with this disclosure is the unsaturated linear and branched hydrocarbons having at least one double bond or triple bond. The linear and branched hydrocarbons include -ene and -yne compounds having two or more carbon atoms. In certain embodiments, the compounds have two to twenty carbon atoms. Examples of unsaturated linear and branched hydrocarbons that may serve as acetylene stabilizers include butene, butadiene, 1-butyne, propyne, pentene, octene, heptene, hexyne, 1-heptyne, 1-octyne, 1-nonyne, 1-decyne, and substituted derivatives of any of these. In certain embodiments, the unsaturated linear and branched hydrocarbon has a vapor pressure of about 700 Torr or lower at 25° C. In certain embodiments, the unsaturated linear and branched hydrocarbon has a vapor pressure of about 100 Torr or lower at 25° C.

Another class of acetylene stabilizer for use with this disclosure is the unsaturated ring hydrocarbons having at least one double bond or triple bond. In certain embodiments, the unsaturated ring hydrocarbon is an aromatic compound having five or more carbon atoms. In certain embodiments, the aromatic hydrocarbon has a vapor pressure of about 700 Torr or lower at 25° C. In certain embodiments, the aromatic hydrocarbon has a vapor pressure of about 5 Torr or lower at 25° C.

Another class of acetylene stabilizer for use with this disclosure is the non-aromatic unsaturated ring hydrocarbons having at least one double bond or triple bond. In certain embodiments, an unsaturated ring hydrocarbon has five or more carbon atoms. Examples include cyclopentene cyclohexene, and substituted derivatives of these. In certain embodiments, the non-aromatic unsaturated ring hydrocarbon has a vapor pressure of about 700 Torr or lower at 25° C. In certain embodiments, the non-aromatic unsaturated ring hydrocarbon has a vapor pressure of about 100 Torr or lower at 25° C.

Acetylene stabilizers may include either one of the above compounds or some combination of these compounds.

### Further Examples of Acetylene Stabilizers

This section presents various examples of acetylene stabilizers useful in embodiments of this disclosure. Some, but not all, the stabilizers described in this section overlap with those presented in the prior section.

The term “acyl,” or “alkanoyl,” as used interchangeably herein, represents groups of 1, 2, 3, 4, 5, 6, 7, 8 or more carbon atoms of a straight, branched, cyclic configuration, saturated, unsaturated and aromatic, and combinations thereof, or hydrogen, attached to the parent molecular group through a carbonyl group, as defined herein. This group is exemplified by formyl, acetyl, propionyl, isobutyryl, butanoyl, and the like. In some embodiments, the acyl or alkanoyl group is —C(O)—R, in which R is hydrogen, an aliphatic group, or an aromatic group, as defined herein.

By “acyl halide” is meant —C(O)X, where X is a halogen, such as Br, F, I, or Cl.

By “aldehyde” is meant a —C(O)H group or a compound including such a group. An example of an aldehyde can include RC(O)H, in which R is selected from aliphatic, haloaliphatic, haloheteroaliphatic, heteroaliphatic, aromatic, aliphatic-aromatic, heteroaliphatic-aromatic, as defined herein, or any combination thereof.

By “aldehydealkyl” is meant an aldehyde group, as defined herein, attached to the parent molecular group through an alkyl group, as defined herein. In some embodiments, the aldehydealkyl group is -L-C(O)H, in which L is an alkyl group, as defined herein.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

By “aliphatic-aryl” is meant an aryl group that is or can be coupled to a compound disclosed herein, where the aryl group is or becomes coupled through an aliphatic group, as defined herein. In some embodiments, the aliphatic-aryl group is -L-R, in which L is an aliphatic group, as defined herein, and R is an aryl group, as defined herein.

By “aliphatic-heteroaryl” is meant a heteroaryl group that is or can be coupled to a compound disclosed herein, wherein the heteroaryl group is or becomes coupled through an aliphatic group, as defined herein. In some embodiments, the aliphatic-heteroaryl group is -L-R, in which L is an aliphatic group, as defined herein, and R is a heteroaryl group, as defined herein.

By “alkyl-aryl,” “alkenyl-aryl,” and “alkynyl-aryl” is meant an aryl group, as defined herein, that is or can be coupled (or attached) to the parent molecular group through an alkyl, alkenyl, or alkynyl group, respectively, as defined herein. The alkyl-aryl, alkenyl-aryl, and/or alkynyl-aryl group can be substituted or unsubstituted. For example, the alkyl-aryl, alkenyl-aryl, and/or alkynyl-aryl group can be substituted with one or more substitution groups, as described herein for alkyl, alkenyl, alkynyl, and/or aryl. Exemplary unsubstituted alkyl-aryl groups are of from 7 to 16 carbons (C₇₋₁₆ alkyl-aryl), as well as those having an alkyl group with 1 to 6 carbons and an aryl group with 4 to 18 carbons (i.e., C₁₋₆ alkyl-C₄₋₁₈ aryl). Exemplary unsubstituted alkenyl-aryl groups are of from 7 to 16 carbons (C₇₋₁₆ alkenyl-aryl), as well as those having an alkenyl group with 2 to 6 carbons and an aryl group with 4 to 18 carbons (i.e., C₂₋₆ alkenyl-C₄₋₁₈ aryl). Exemplary unsubstituted alkynyl-aryl groups are of from 7 to 16 carbons (C₇₋₁₆ alkynyl-aryl), as well as those having an alkynyl group with 2 to 6 carbons and an aryl group with 4 to 18 carbons (i.e., C₂₋₆ alkynyl-C₄₋₁₈ aryl). In some embodiments, the alkyl-aryl group is -L-R, in which L is an alkyl group, as defined herein, and R is an aryl group, as defined herein. In some embodiments, the alkenyl-aryl group is -L-R, in which L is an alkenyl group, as defined herein, and R is an aryl group, as defined herein. In some embodiments, the alkynyl-aryl group is -L-R, in which L is an alkynyl group, as defined herein, and R is an aryl group, as defined herein.

By “alkyl-cycloalkyl” is meant a cycloalkyl group, as defined herein, attached to the parent molecular group through an alkyl group, as defined herein. The alkyl-cycloalkyl group can be substituted or unsubstituted. For example, the alkyl-cycloalkyl group can be substituted with one or more substitution groups, as described herein for alkyl. In some embodiments, the alkyl-cycloalkyl group is -L-R, in which L is an alkyl group, as defined herein, and R is a cycloalkyl group, as defined herein.

By “alkenyl” is meant an unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). An exemplary alkenyl includes an optionally substituted C₂₋₂₄ alkyl group having one or more double bonds. The alkenyl group can be monovalent or multivalent (e.g., bivalent) by removing one or more hydrogens to form appropriate attachment to the parent molecular group or appropriate attachment between the parent molecular group and another substitution. The alkenyl group can also be substituted or unsubstituted. For example, the alkenyl group can be substituted with one or more substitution groups, as described herein for alkyl.

By “alkyl-heteroaryl” is meant a heteroaryl group, as defined herein, attached to the parent molecular group through an alkyl group, as defined herein. In some embodiments, the alkyl-heteroaryl group is -L-R, in which L is an alkyl group, as defined herein, and R is a heteroaryl group, as defined herein.

By “alkyl-heterocyclyl,” “alkenyl-heterocyclyl,” and “alkynyl-heterocyclyl” is meant a heterocyclyl group, as defined herein, that is or can be coupled (or attached) to the parent molecular group through an alkyl, alkenyl, or alkynyl group, respectively, as defined herein. The alkyl-heterocyclyl, alkenyl-heterocyclyl, and/or alkynyl-heterocyclyl group can be substituted or unsubstituted. For example, the alkyl-heterocyclyl, alkenyl-heterocyclyl, and/or alkynyl-heterocyclyl group can be substituted with one or more substitution groups, as described herein for alkyl, alkenyl, alkynyl, and/or heterocyclyl. Exemplary unsubstituted alkyl-heterocyclyl groups are of from 2 to 16 carbons (C₂₋₁₆ alkyl-heterocyclyl), as well as those having an alkyl group with 1 to 6 carbons and a heterocyclyl group with 1 to 18 carbons (i.e., C₁₋₆ alkyl-C₁₋₁₈ heterocyclyl). Exemplary unsubstituted alkenyl-heterocyclyl groups are of from 3 to 16 carbons (C₃₋₁₆ alkenyl-heterocyclyl), as well as those having an alkenyl group with 2 to 6 carbons and a heterocyclyl group with 1 to 18 carbons (i.e., C₂₋₆ alkenyl-C₁₋₁₈ heterocyclyl). Exemplary unsubstituted alkynyl-heterocyclyl groups are of from 3 to 16 carbons (C₃₋₁₆ alkynyl-heterocyclyl), as well as those having an alkynyl group with 2 to 6 carbons and a heterocyclyl group with 1 to 18 carbons (i.e., C₂₋₆ alkynyl-C₁₋₁₈ heterocyclyl). In some embodiments, the alkyl-heterocyclyl group is -L-R, in which L is an alkyl group, as defined herein, and R is a heterocyclyl group, as defined herein. In some embodiments, the alkenyl-heterocyclyl group is -L-R, in which L is an alkenyl group, as defined herein, and R is a heterocyclyl group, as defined herein. In some embodiments, the alkynyl-heterocyclyl group is -L-R, in which L is an alkynyl group, as defined herein, and R is a heterocyclyl group, as defined herein.

By “alkoxy” is meant —OR, where R is an optionally substituted aliphatic group, as described herein. Exemplary alkoxy groups include, but are not limited to, methoxy, ethoxy, n-propoxy, isopropoxy, n-butoxy, t-butoxy, sec-butoxy, n-pentoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkoxyalkyl” is meant an alkyl group, as defined herein, which is substituted with an alkoxy group, as defined herein. Exemplary unsubstituted alkoxyalkyl groups include between 2 to 12 carbons (C₂₋₁₂ alkoxyalkyl), as well as those having an alkyl group with 1 to 6 carbons and an alkoxy group with 1 to 6 carbons (i.e., C₁₋₆ alkoxy-C₁₋₆ alkyl). In some embodiments, the alkoxyalkyl group is -L-O—R, in which each of L and R is, independently, an alkyl group, as defined herein.

By “alkyl” is meant a saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl). An exemplary alkyl includes a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be monovalent or multivalent (e.g., bivalent) by removing one or more hydrogens to form appropriate attachment to the parent molecular group or appropriate attachment between the parent molecular group and another substitution. For example, the alkyl group can be substituted with one, two, three or, in the case of alkyl groups of two carbons or more, four substituents independently selected from the group consisting of: (1) C₁₋₆ alkoxy (e.g., —O—R, in which R is C₁₋₆ alkyl); (2) C₁₋₆ alkylsulfinyl (e.g., —S(O)—R, in which R is C₁₋₆ alkyl); (3) C₁₋₆ alkylsulfonyl (e.g., —SO₂—R, in which R is C₁₋₆ alkyl); (4) amine (e.g., —C(O)NR¹R² or —NHCOR¹, where each of R¹ and R² is, independently, selected from hydrogen, aliphatic, heteroaliphatic, haloaliphatic, haloheteroaliphatic, aromatic, as defined herein, or any combination thereof, or R¹ and R², taken together with the nitrogen atom to which each are attached, can form a heterocyclyl group, as defined herein); (5) aryl; (6) arylalkoxy (e.g., —O-L-R, in which L is alkyl and R is aryl); (7) aryloyl (e.g., —C(O)—R, in which R is aryl); (8) azido (e.g., —N₃); (9) cyano (e.g., —CN); (10) aldehyde (e.g., —C(O)H); (11) C₃₋₈ cycloalkyl; (12) halo; (13) heterocyclyl (e.g., as defined herein, such as a 5-, 6- or 7-membered ring containing one, two, three, or four non-carbon heteroatoms); (14) heterocyclyloxy (e.g., —O—R, in which R is heterocyclyl, as defined herein): (15) heterocyclyloyl (e.g., —C(O)—R, in which R is heterocyclyl, as defined herein); (16) hydroxyl (e.g., —OH); (17) N-protected amino; (18) nitro (e.g., —NO₂); (19) oxo (e.g., ═O); (20) C₁₋₆ thioalkoxy (e.g., —S—R, in which R is alkyl); (21) thiol (e.g., —SH); (22) —CO₂R¹, where R¹ is selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (23) —C(O)NR¹R², where each of R¹ and R² is, independently, selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (24) —SO₂R¹, where R¹ is selected from the group consisting of (a) C₁₋₆ alkyl, (b) C₄₋₁₈ aryl, and (c) C₁₋₆ alkyl-C₄₋₁₈ aryl (e.g., -L-R, in which L is C₁₋₆ alkyl and R is C₄₋₁₈ aryl); (25) —SO₂NR¹R², where each of R¹ and R² is, independently, selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); and (26) —NR¹R², where each of R¹ and R² is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alkyl-C₄₋₁₈ aryl (e.g., -L-R, in which L is C₁₋₆ alkyl and R is C₄₋₁₈ aryl), (h) C₃₋₈ cycloalkyl, and (i) C₁₋₆ alkyl-C₃₋₈ cycloalkyl (e.g., -L-R, in which L is C₁₋₆ alkyl and R is C₃₋₈ cycloalkyl), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a bivalent form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂), C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkylsulfinyl” is meant an alkyl group, as defined herein, attached to the parent molecular group through an —S(O)— group. In some embodiments, the unsubstituted alkylsulfinyl group is a C₁₋₆ or C₁₋₁₂ alkylsulfinyl group. In other embodiments, the alkylsulfinyl group is —S(O)—R, in which R is an alkyl group, as defined herein.

By “alkylsulfinylalkyl” is meant an alkyl group, as defined herein, substituted by an alkylsulfinyl group. In some embodiments, the unsubstituted alkylsulfinylalkyl group is a C₂₋₁₂ or C₂₋₂₄ alkylsulfinylalkyl group (e.g., C₁₋₆ alkylsulfinyl-C₁₋₆ alkyl or C₁₋₁₂ alkylsulfinyl-C₁₋₁₂ alkyl). In other embodiments, the alkylsulfinylalkyl group is -L-S(O)—R, in which each of L and R is, independently, an alkyl group, as defined herein.

By “alkylsulfonyl” is meant an alkyl group, as defined herein, attached to the parent molecular group through an —SO₂— group. In some embodiments, the unsubstituted alkylsulfonyl group is a C₁₋₆ or C₁₋₁₂ alkylsulfonyl group. In other embodiments, the alkylsulfonyl group is —SO₂—R, where R is an optionally substituted alkyl (e.g., as described herein, including optionally substituted C₁₋₁₂ alkyl, haloalkyl, or perfluoroalkyl).

By “alkylsulfonylalkyl” is meant an alkyl group, as defined herein, substituted by an alkylsulfonyl group. In some embodiments, the unsubstituted alkylsulfonylalkyl group is a C₂₋₁₂ or C₂₋₂₄ alkylsulfonylalkyl group (e.g., C₁₋₆ alkylsulfonyl-C₁₋₁₂ alkyl or C₁₋₁₂ alkylsulfonyl-C₁₋₁₂ alkyl). In other embodiments, the alkylsulfonylalkyl group is -L-SO₂—R, in which each of L and R is, independently, an alkyl group, as defined herein.

By “alkynyl” is meant an unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). An exemplary alkynyl includes an optionally substituted C₂₋₂₄ alkyl group having one or more triple bonds. The alkynyl group can be cyclic or acyclic and is exemplified by ethynyl, 1-propynyl, and the like. The alkynyl group can be monovalent or multivalent (e.g., bivalent) by removing one or more hydrogens to form appropriate attachment to the parent molecular group or appropriate attachment between the parent molecular group and another substitution. The alkynyl group can also be substituted or unsubstituted. For example, the alkynyl group can be substituted with one or more substitution groups, as described herein for alkyl.

By “ambient temperature” is meant a temperature ranging from 16° C. to 26° C., such as from 19° C. to 25° C. or from 20° C. to 25° C.

By “amide” is mean —C(O)NR¹R² or —NHCOR¹, where each of R¹ and R² is, independently, selected from hydrogen, aliphatic, heteroaliphatic, haloaliphatic, haloheteroaliphatic, aromatic, as defined herein, or any combination thereof, or where R¹ and R², taken together with the nitrogen atom to which each are attached, can form a heterocyclyl group, as defined herein.

By “amine” is meant —NR¹R², where each of R¹ and R² is, independently, selected from hydrogen, aliphatic, heteroaliphatic, haloaliphatic, haloheteroaliphatic, aromatic, as defined herein, or any combination thereof; or where R¹ and R², taken together with the nitrogen atom to which each are attached, can form a heterocyclyl group, as defined herein.

By “aminoalkyl” is meant an alkyl group, as defined herein, substituted by an amine group, as defined herein. In some embodiments, the aminoalkyl group is -L-NR¹R², in which L is an alkyl group, as defined herein, and each of R¹ and R² is, independently, selected from hydrogen, aliphatic, heteroaliphatic, haloaliphatic, haloheteroaliphatic, aromatic, as defined herein, or any combination thereof; or R¹ and R², taken together with the nitrogen atom to which each are attached, can form a heterocyclyl group, as defined herein. In other embodiments, the aminoalkyl group is -L-C(NR¹R²)(R³)—R⁴, in which L is a covalent bond or an alkyl group, as defined herein; each of R¹ and R² is, independently, selected from hydrogen, aliphatic, heteroaliphatic, haloaliphatic, haloheteroaliphatic, aromatic, as defined herein, or any combination thereof; or R¹ and R², taken together with the nitrogen atom to which each are attached, can form a heterocyclyl group, as defined herein; and each of R¹ and R⁴ is, independently, H or alkyl, as defined herein.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane x-electrons corresponds to the Huckel rule (4n+2). The point of attachment to the parent structure typically is through an aromatic portion of the condensed ring system.

By “aryl” is meant an aromatic carbocyclic group comprising at least five carbon atoms to 15 carbon atoms (C₅₋₁₅), such as five to ten carbon atoms (C₅₋₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, aromatic, other functional groups, or any combination thereof. Exemplary aryl groups include, but are not limited to, benzyl, naphthalene, phenyl, biphenyl, phenoxybenzene, and the like. The term aryl also includes heteroaryl, which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)—R, in which R is C₁₋₆ alkyl); (2) C₁₋₆ alkyl; (3) C₁₋₆ alkoxy (e.g., —O—R, in which R is C₁₋₆ alkyl); (4) C₁₋₆ alkoxy-C₁₋₆ alkyl (e.g., -L-O—R, in which each of L and R is, independently, C₁₋₆ alkyl); (5) C₁₋₆ alkylsulfinyl (e.g., —S(O)—R, in which R is C₁₋₆ alkyl); (6) C₁₋₆ alkylsulfinyl-C₁₋₆ alkyl (e.g., -L-S(O)—R, in which each of L and R is, independently, C₁₋₆ alkyl); (7) C₁₋₆ alkylsulfonyl (e.g., —SO₂—R, in which R is C₁₋₆ alkyl); (8) C₁₋₆ alkylsulfonyl-C₁₋₆ alkyl (e.g., -L-SO₂—R, in which each of L and R is, independently, C₁₋₆ alkyl); (9) aryl; (10) amine (e.g., —NR¹R², where each of R¹ and R² is, independently, selected from hydrogen, aliphatic, heteroaliphatic, haloaliphatic, haloheteroaliphatic, aromatic, as defined herein, or any combination thereof; or R¹ and R², taken together with the nitrogen atom to which each are attached, can form a heterocyclyl group, as defined herein); (11) C₁₋₆ aminoalkyl (e.g., -L¹-NR¹R² or -L²-C(NR¹R²)(R³)—R⁴, in which L¹ is C₁₋₆ alkyl; L² is a covalent bond or C₁₋₆ alkyl; each of R¹ and R² is, independently, selected from hydrogen, aliphatic, heteroaliphatic, haloaliphatic, haloheteroaliphatic, aromatic, as defined herein, or any combination thereof; or R¹ and R², taken together with the nitrogen atom to which each are attached, can form a heterocyclyl group, as defined herein; and each of R³ and R⁴ is, independently, H or C₁₋₆ alkyl); (12) heteroaryl; (13) C₁₋₆ alkyl-C₄₋₁₈ aryl (e.g., -L-R, in which L is C₁₋₆ alkyl and R is C₄₋₁₈ aryl); (14) aryloyl (e.g., —C(O)—R, in which R is aryl); (15) azido (e.g., —N₃); (16) cyano (e.g., —CN); (17) C₁₋₆ azidoalkyl (e.g., -L-N₃, in which L is C₁₋₆ alkyl); (18) aldehyde (e.g., —C(O)H); (19) aldehyde-C₁₋₆ alkyl (e.g., -L-C(O)H, in which L is C₁₋₆ alkyl); (20) C₃₋₈ cycloalkyl; (21) C₁₋₆ alkyl-C₃₋₈ cycloalkyl (e.g., -L-R, in which L is C₁₋₆ alkyl and R is C₃₋₈ cycloalkyl); (22) halo; (23) C₁₋₆ haloalkyl (e.g., -L¹-X or -L²-C(X)(R¹)—R², in which L¹ is C₁₋₆ alkyl; L² is a covalent bond or C₁₋₆ alkyl; X is fluoro, bromo, chloro, or iodo; and each of R¹ and R² is, independently, H or C₁₋₆ alkyl); (24) heterocyclyl (e.g., as defined herein, such as a 5-, 6- or 7-membered ring containing one, two, three, or four non-carbon heteroatoms); (25) heterocyclyloxy (e.g., —O—R, in which R is heterocyclyl, as defined herein); (26) heterocyclyloyl (e.g., —C(O)—R, in which R is heterocyclyl, as defined herein); (27) hydroxyl (—OH); (28) C₁₋₆ hydroxyalkyl (e.g., -L¹-OH or -L²-C(OH)(R¹)—R², in which L¹ is C₁₋₆ alkyl; L² is a covalent bond or alkyl; and each of R¹ and R² is, independently, H or C₁₋₆ alkyl, as defined herein); (29) nitro; (30) C₁₋₆ nitroalkyl (e.g., -L¹-NO or -L²-C(NO)(R¹)—R², in which L¹ is C₁₋₆ alkyl; L² is a covalent bond or alkyl; and each of R¹ and R² is, independently, H or C₁₋₆ alkyl, as defined herein); (31) N-protected amino; (32) N-protected amino-C₁₋₆ alkyl; (33) oxo (e.g., ═O); (34) C₁₋₆ thioalkoxy (e.g., —S—R, in which R is C₁₋₆ alkyl); (35) thio-C₁₋₆ alkoxy-C₁₋₆ alkyl (e.g., -L-S—R, in which each of L and R is, independently, C₁₋₆ alkyl); (36) —(CH₂)ᵣCO₂R¹, where r is an integer of from zero to four, and R¹ is selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (37) —(CH₂)ᵣCONR¹R², where r is an integer of from zero to four and where each R¹ and R² is independently selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (38) —(CH₂)ᵣSO₂R¹, where r is an integer of from zero to four and where R¹ is selected from the group consisting of (a) C₁₋₆ alkyl, (b) C₄₋₁₈ aryl, and (c) C₁₋₆ alkyl-C₄₋₁₈ aryl (e.g., -L-R, in which L is C₁₋₆ alkyl and R is C₄₋₁₈ aryl); (39) —(CH₂)ᵣSO₂NR¹R², where r is an integer of from zero to four and where each of R¹ and R² is, independently, selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (40) —(CH₂)ᵣNR¹R², where r is an integer of from zero to four and where each of R¹ and R² is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alkyl-C₄₋₁₈ aryl (e.g., -L-R, in which L is C₁₋₆ alkyl and R is C₄₋₁₈ aryl), (h) C₃₋₈ cycloalkyl, and (i) C₁₋₆ alkyl-C₃₋₈ cycloalkyl (e.g., -L-R, in which L is C₁₋₆ alkyl and R is C₃₋₈ cycloalkyl), wherein in one embodiment no two groups are bound to the nitrogen atom through a carbonyl group or a sulfonyl group; (41) thiol (e.g., —SH); (42) perfluoroalkyl (e.g., —(CF₂)ₙCF₃, in which n is an integer from 0 to 10); (43) perfluoroalkoxy (e.g., —O—(CF₂)ₙCF₃, in which n is an integer from 0 to 10); (44) aryloxy (e.g., —O—R, in which R is aryl); (45) cycloalkoxy (e.g., —O—R, in which R is cycloalkyl); (46) cycloalkylalkoxy (e.g., —O-L-R, in which L is alkyl and R is cycloalkyl); and (47) arylalkoxy (e.g., —O-L-R, in which L is alkyl and R is aryl). In particular embodiments, an unsubstituted aryl group is a C₄₋₁₈, C₄₋₁₄, C₄₋₁₂, C₄₋₁₀, C₆₋₁₅, C₆₋₁₄, C₆₋₁₂, or C₆₋₁₀ aryl group.

By “arylalkoxy” is meant an alkyl-aryl group, as defined herein, attached to the parent molecular group through an oxygen atom. In some embodiments, the arylalkoxy group is —O-L-R, in which L is an alkyl group, as defined herein, and R is an aryl group, as defined herein.

By “aryloxy” is meant —OR, where R is an optionally substituted aryl group, as described herein. In some embodiments, an unsubstituted aryloxy group is a C₄₋₁₈ or C₆₋₁₈ aryloxy group.

By “aryloxycarbonyl” is meant an aryloxy group, as defined herein, that is attached to the parent molecular group through a carbonyl group. In some embodiments, an unsubstituted aryloxycarbonyl group is a C₅₋₁₉ aryloxycarbonyl group. In other embodiments, the aryloxycarbonyl group is —C(O)O—R, in which R is an aryl group, as defined herein.

By “aryloyl” is meant an aryl group that is attached to the parent molecular group through a carbonyl group. In some embodiments, an unsubstituted aryloyl group is a C₇₋₁₁ aryloyl or C₅₋₁₉ aryloyl group. In other embodiments, the aryloyl group is —C(O)—R, in which R is an aryl group, as defined herein.

By “azido” is meant an —N₃ group.

By “azidoalkyl” is meant an azido group attached to the parent molecular group through an alkyl group, as defined herein. In some embodiments, the azidoalkyl group is -L-N₃, in which L is an alkyl group, as defined herein.

By “azo” is meant an —N═N— group.

By “carbene” is meant H₂C⁺ and derivatives thereof having carbon bearing two nonbonding electrons or (C:). In some embodiments, the carbene is R¹R²(C:), where each of R¹ and R² is, independently, selected from hydrogen, aliphatic, heteroaliphatic, haloaliphatic, haloheteroaliphatic, aromatic, as defined herein, or any combination thereof, or R¹ and R², taken together with the atom to which each are attached, form a cycloaliphatic group, as defined herein.

By “carbenium cation” is meant H₃C⁺ and derivatives thereof having carbon bearing a +1 formal charge or C*. In some embodiments, the carbenium cation is R¹—C⁺(R)—R², where each of R, R¹, and R² is, independently, selected from hydrogen, aliphatic, heteroaliphatic, haloaliphatic, haloheteroaliphatic, aromatic, as defined herein, or any combination thereof, or R¹ and R² and optionally R, taken together with the atom to which each are attached, form a cycloaliphatic group, as defined herein.

By “carbonyl” is meant a —C(O)— group, which can also be represented as >C═O.

By “carboxyl” is meant a —CO₂H group or an anion thereof.

By “catalyst” is meant a compound, usually present in small amounts relative to reactants, capable of catalyzing a synthetic reaction, as would be readily understood by a person of ordinary skill in the art. In some embodiments, catalysts may include transition metal coordination complex.

By “cyano” is meant a —CN group.

By “cycloaliphatic” is meant an aliphatic group, as defined herein, that is cyclic.

By “cycloalkoxy” is meant a cycloalkyl group, as defined herein, attached to the parent molecular group through an oxygen atom. In some embodiments, the cycloalkoxy group is —O—R, in which R is a cycloalkyl group, as defined herein.

By “cycloalkylalkoxy” is meant an alkyl-cycloalkyl group, as defined herein, attached to the parent molecular group through an oxygen atom. In some embodiments, the cycloalkylalkoxy group is —O-L-R, in which L is an alkyl group, as defined herein, and R is a cycloalkyl group, as defined herein.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.heptyl], and the like. The cycloalkyl group can also be substituted or unsubstituted. For example, the cycloalkyl group can be substituted with one or more groups including those described herein for alkyl.

By “cycloheteroaliphatic” is meant a heteroaliphatic group, as defined herein, that is cyclic.

By “disulfide” is meant —SSR, where R is selected from hydrogen, aliphatic, heteroaliphatic, haloaliphatic, haloheteroaliphatic, aromatic, as defined herein, or any combination thereof.

By “electron-donating group” is meant a functional group capable of donating at least a portion of its electron density into the ring to which it is directly attached, such as by resonance.

By “electron-withdrawing group” is meant a functional group capable of accepting electron density from the ring to which it is directly attached, such as by inductive electron withdrawal.

By “ester” is meant —C(O)OR or —OC(O)R, where R is selected from aliphatic, heteroaliphatic, haloaliphatic, haloheteroaliphatic, aromatic, as defined herein, or any combination thereof.

By “halo” is meant F, Cl, Br, or I.

By “haloaliphatic” is meant an aliphatic group, as defined herein, in which one or more hydrogen atoms, such as one to 10 hydrogen atoms, independently is replaced with a halogen atom, such as fluoro, bromo, chloro, or iodo.

By “haloaliphatic-aryl” is meant an aryl group, as defined herein, that is or can be coupled to a compound disclosed herein, where the aryl group is or becomes coupled through a haloaliphatic group, as defined herein. In some embodiments, the haloaliphatic-aryl group is -L-R, in which L is a haloaliphatic group, as defined herein, and R is an aryl group, as defined herein.

By “haloaliphatic-heteroaryl” is meant an heteroaryl group, as defined herein, that is or can be coupled to a compound disclosed herein, where the heteroaryl group is or becomes coupled through a haloaliphatic group, as defined herein. In some embodiments, the haloaliphatic-heteroaryl group is -L-R, in which L is a haloaliphatic group, as defined herein, and R is a heteroaryl group, as defined herein.

By “haloalkyl” is meant an alkyl group, as defined herein, where one or more hydrogen atoms, such as one to 10 hydrogen atoms, independently is replaced with a halogen atom, such as fluoro, bromo, chloro, or iodo. In an independent embodiment, haloalkyl can be a —CX₃ group, wherein each X independently can be selected from fluoro, bromo, chloro, or iodo. In some embodiments, the haloalkyl group is -L-X, in which L is an alkyl group, as defined herein, and X is fluoro, bromo, chloro, or iodo. In other embodiments, the haloalkyl group is -L-C(X)(R¹)—R², in which L is a covalent bond or an alkyl group, as defined herein; X is fluoro, bromo, chloro, or iodo; and each of R¹ and R² is, independently, H or alkyl, as defined herein.

By “haloheteroaliphatic” is meant a heteroaliphatic, as defined herein, in which one or more hydrogen atoms, such as one to 10 hydrogen atoms, independently is replaced with a halogen atom, such as fluoro, bromo, chloro, or iodo.

By “heteroaliphatic” is meant an aliphatic group, as defined herein, including at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

By “heteroaliphatic-aryl” is meant an aryl group, as defined herein, that is or can be coupled to a compound disclosed herein, where the aryl group is or becomes coupled through a heteroaliphatic group, as defined herein. In some embodiments, the heteroaliphatic-aryl group is -L-R, in which L is a heteroaliphatic group, as defined herein, and R is an aryl group, as defined herein.

By “heteroalkyl,” “heteroalkenyl,” and “heteroalkynyl” is meant an alkyl, alkenyl, or alkynyl group (which can be branched, straight-chain, or cyclic), respectively, as defined herein, including at least one heteroatom to 20 heteroatoms, such as one to 15 heteroatoms, or one to 5 heteroatoms, which can be selected from, but not limited to, oxygen, nitrogen, sulfur, silicon, boron, selenium, phosphorous, and oxidized forms thereof within the group.

By “heteroalkyl-aryl,” “heteroalkenyl-aryl,” and “heteroalkynyl-aryl” is meant an aryl group, as defined herein, that is or can be coupled to a compound disclosed herein, where the aryl group is or becomes coupled through a heteroalkyl, heteroalkenyl, or heteroalkynyl group, respectively, as defined herein. In some embodiments, the heteroalkyl-aryl group is -L-R, in which L is a heteroalkyl group, as defined herein, and R is an aryl group, as defined herein. In some embodiments, the heteroalkenyl-aryl group is -L-R, in which L is a heteroalkenyl group, as defined herein, and R is an aryl group, as defined herein. In some embodiments, the heteroalkynyl-aryl group is -L-R, in which L is a heteroalkynyl group, as defined herein, and R is an aryl group, as defined herein.

By “heteroalkyl-heteroaryl,” “heteroalkenyl-heteroaryl,” and “heteroalkynyl-heteroaryl” is meant a heteroaryl group, as defined herein, that is or can be coupled to a compound disclosed herein, where the heteroaryl group is or becomes coupled through a heteroalkyl, heteroalkenyl, or heteroalkynyl group, respectively, as defined herein. In some embodiments, the heteroalkyl-heteroaryl group is -L-R, in which L is a heteroalkyl group, as defined herein, and R is a heteroaryl group, as defined herein. In some embodiments, the heteroalkenyl-heteroaryl group is -L-R, in which L is a heteroalkenyl group, as defined herein, and R is a heteroaryl group, as defined herein. In some embodiments, the heteroalkynyl-heteroaryl group is -L-R, in which L is a heteroalkynyl group, as defined herein, and R is a heteroaryl group, as defined herein.

By “heteroaryl” is meant an aryl group including at least one heteroatom to six heteroatoms, such as one to four heteroatoms, which can be selected from, but not limited to, oxygen, nitrogen, sulfur, silicon, boron, selenium, phosphorous, and oxidized forms thereof within the ring. Such heteroaryl groups can have a single ring or multiple condensed rings, where the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, aromatic, other functional groups, or any combination thereof. An exemplary heteroaryl includes a subset of heterocyclyl groups, as defined herein, which are aromatic, i.e., they contain 4n+2 pi electrons within the mono- or multicyclic ring system.

By “heteroatom” is meant an atom other than carbon, such as oxygen, nitrogen, sulfur, silicon, boron, selenium, or phosphorous. In particular disclosed embodiments, such as when valency constraints do not permit, a heteroatom does not include a halogen atom.

By “heterocyclyl” is meant a 5-, 6- or 7-membered ring, unless otherwise specified, containing one, two, three, or four non-carbon heteroatoms (e.g., independently selected from the group consisting of nitrogen, oxygen, phosphorous, sulfur, or halo). The 5-membered ring has zero to two double bonds and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include thiiranyl, thietanyl, tetrahydrothienyl, thianyl, thiepanyl, aziridinyl, azetidinyl, pyrrolidinyl, piperidinyl, azepanyl, pyrrolyl, pyrrolinyl, pyrazolyl, pyrazolinyl, pyrazolidinyl, imidazolyl, imidazolinyl, imidazolidinyl, pyridyl, homopiperidinyl, pyrazinyl, piperazinyl, pyrimidinyl, pyridazinyl, oxazolyl, oxazolidinyl, oxazolidonyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, dihydrothienyl, dihydroindolyl, tetrahydroquinolyl, tetrahydroisoquinolyl, pyranyl, dihydropyranyl, tetrahydropyranyl, dithiazolyl, dioxanyl, dioxinyl, dithianyl, trithianyl, oxazinyl, thiazinyl, oxothiolanyl, triazinyl, benzofuranyl, benzothienyl, and the like.

By “heterocyclyloxy” is meant a heterocyclyl group, as defined herein, attached to the parent molecular group through an oxygen atom. In some embodiments, the heterocyclyloxy group is —O—R, in which R is a heterocyclyl group, as defined herein.

By “heterocyclyloyl” is meant a heterocyclyl group, as defined herein, attached to the parent molecular group through a carbonyl group. In some embodiments, the heterocyclyloyl group is —C(O)—R, in which R is a heterocyclyl group, as defined herein.

By “hydroxyl” is meant —OH.

By “hydroxyalkyl” is meant an alkyl group, as defined herein, substituted by one to three hydroxyl groups, with the proviso that no more than one hydroxyl group may be attached to a single carbon atom of the alkyl group and is exemplified by hydroxymethyl, dihydroxypropyl, and the like. In some embodiments, the hydroxyalkyl group is -L-OH, in which L is an alkyl group, as defined herein. In other embodiments, the hydroxyalkyl group is -L-C(OH)(R¹)—R², in which L is a covalent bond or an alkyl group, as defined herein, and each of R¹ and R² is, independently, H or alkyl, as defined herein.

By “ketone” is meant —C(O)R or a compound including such a group, where R is selected from aliphatic, heteroaliphatic, aromatic, as defined herein, or any combination thereof. An example of a ketone can include R¹C(O)R, in which each of R and R¹ is, independently, selected from aliphatic, haloaliphatic, haloheteroaliphatic, heteroaliphatic, aromatic, aliphatic-aromatic, heteroaliphatic-aromatic, as defined herein, or any combination thereof.

By “nitro” is meant an —NO₂ group.

By “nitroalkyl” is meant an alkyl group, as defined herein, substituted by one to three nitro groups. In some embodiments, the nitroalkyl group is -L-NO, in which L is an alkyl group, as defined herein. In other embodiments, the nitroalkyl group is -L-C(NO)(R¹)—R², in which L is a covalent bond or an alkyl group, as defined herein, and each of R¹ and R² is, independently, H or alkyl, as defined herein.

By “oxo” is meant an ═O group.

By “oxy” is meant —O—.

By “perfluoroalkyl” is meant an alkyl group, as defined herein, having each hydrogen atom substituted with a fluorine atom. Exemplary perfluoroalkyl groups include trifluoromethyl, pentafluoroethyl, etc. In some embodiments, the perfluoroalkyl group is —(CF₂)ₙCF₃, in which n is an integer from 0 to 10.

By “perfluoroalkoxy” is meant an alkoxy group, as defined herein, having each hydrogen atom substituted with a fluorine atom. In some embodiments, the perfluoroalkoxy group is —O—R, in which R is a perfluoroalkyl group, as defined herein.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S. M. et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, gluconate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include ammonium, sulfonium, sulfoxonium, phosphonium, iminium, imidazolium, benzimidazolium, amidinium, guanidinium, phosphazinium, phosphazenium, pyridinium, etc., as well as other cationic groups described herein (e.g., optionally substituted isoxazolium, optionally substituted oxazolium, optionally substituted thiazolium, optionally substituted pyrrolium, optionally substituted furanium, optionally substituted thiophenium, optionally substituted imidazolium, optionally substituted pyrazolium, optionally substituted isothiazolium, optionally substituted triazolium, optionally substituted tetrazolium, optionally substituted furazanium, optionally substituted pyridinium, optionally substituted pyrimidinium, optionally substituted pyrazinium, optionally substituted triazinium, optionally substituted tetrazinium, optionally substituted pyridazinium, optionally substituted oxazinium, optionally substituted pyrrolidinium, optionally substituted pyrazolidinium, optionally substituted imidazolinium, optionally substituted isoxazolidinium, optionally substituted oxazolidinium, optionally substituted piperazinium, optionally substituted piperidinium, optionally substituted morpholinium, optionally substituted azepanium, optionally substituted azepinium, optionally substituted indolium, optionally substituted isoindolium, optionally substituted indolizinium, optionally substituted indazolium, optionally substituted benzimidazolium, optionally substituted isoquinolinum, optionally substituted quinolizinium, optionally substituted dehydroquinolizinium, optionally substituted quinolinium, optionally substituted isoindolinium, optionally substituted benzimidazolinium, and optionally substituted purinium).

By “silyl ether” is meant a functional group including a silicon atom covalently bound to an alkoxy group, as defined herein. In some embodiments, the silyl ether is —Si—O—R or Si—O—R, in which R is an alkyl group, as defined herein.

By “sulfinyl” is meant an —S(O)— group.

By “sulfo” is meant an —S(O)₂OH group.

By “sulfonyl” or “sulfonate” is meant an —S(O)₂— group or a —SO₂R, where R is selected from hydrogen, aliphatic, heteroaliphatic, haloaliphatic, haloheteroaliphatic, aromatic, as defined herein, or any combination thereof.

By “thioalkoxy” is meant an alkyl group, as defined herein, attached to the parent molecular group through a sulfur atom. Exemplary unsubstituted thioalkoxy groups include C₁₋₆ thioalkoxy. In some embodiments, the thioalkoxy group is —S—R, in which R is an alkyl group, as defined herein.

By “thioalkoxyalkyl” is meant an alkyl group, as defined herein, which is substituted with a thioalkoxy group, as defined herein. Exemplary unsubstituted thioalkoxyalkyl groups include between 2 to 12 carbons (C₂₋₁₂ thioalkoxyalkyl), as well as those having an alkyl group with 1 to 6 carbons and a thioalkoxy group with 1 to 6 carbons (i.e., C₁₋₆ thioalkoxy-C₁₋₆ alkyl). In some embodiments, the thioalkoxyalkyl group is -L-S—R, in which each of L and R is, independently, an alkyl group, as defined herein.

By “thiol” is meant an —SH group.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated therein.

### Acids

In one embodiment, the stabilizer is an acid having a formula R—CO₂H wherein R is selected from hydrogen, aliphatic, haloaliphatic, haloheteroaliphatic, heteroaliphatic, aromatic, aliphatic-aromatic, heteroaliphatic-aromatic or any combinations thereof. In certain embodiments, R is alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heteroaryl, alkyl-aryl, alkenyl-aryl, alkynyl-aryl, alkyl-heteroaryl, alkenyl-heteroaryl, alkynyl-heteroaryl, heteroalkyl-aryl, heteroalkenyl-aryl, heteroalkynyl-aryl, heteroalkyl-heteroaryl, heteroalkenyl-heteroaryl, heteroalkynyl-heteroaryl or any combinations thereof. In particular disclosed embodiments, R may further be substituted with one or more substituents such as, alkoxy, amide, amine, thioether, hydroxyl, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide or any combinations thereof. In some embodiments, the acid is not a C₂ carboxylic acid (e.g., acetic acid).

### Alcohols

The stabilizer can be an alcohol having a formula of X—C(R)ₙ(OH)—Y, where:
n is 1; and
each X and Y can be independently selected from hydrogen, —[C(R¹)₂]ₘ—C(R¹)₃, or OH, wherein each R¹ is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein m is an integer from 0 to 10; and each R independently is selected from hydrogen, hydroxyl, aliphatic, haloaliphatic, haloheteroaliphatic, heteroaliphatic, aromatic, aliphatic-aromatic, heteroaliphatic-aromatic, or any combinations thereof.

In some embodiments, each R and R¹ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the alcohol may further be substituted with one or more substituents, such as alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when at least one of X or Y=—[C(R¹)₂]ₘ—C(R¹)₃, the stabilizer can be a C₅-C₁₀ alcohol. Exemplary stabilizers include, but are not limited to, C₅-C₁₀ alcohols, such as, 1-pentanol, 2-pentanol, 3-methyl-1-butanol, and the like.

In other embodiments, when at least one of X or Y=—[C(R¹)₂]ₘ—C(R¹)₃ or R is not hydrogen, the stabilizer can be a C₃ alcohol. For instance, if R¹ or R is alkenyl, then the C₃ alcohol can be a C₃ alkenol (e.g., allyl alcohol). In another instance, if R¹ or R is cycloaliphatic, then the C₃ alcohol can be a cyclopropanol or 2-cyclopropenol.

In yet other embodiments, when at least one of X or Y=—[C(R¹)₂]ₘ—C(R¹)₃ or R is not hydrogen, the stabilizer can be a C₄ alcohol. For instance, if R¹ or R is alkenyl, then the C₄ alcohol can be a C₄ alkenol (e.g., 2-buten-1-ol or 3-buten-1-ol). In another instance, if R¹ or R is cycloaliphatic, then the C₄ alcohol can be a C₄-cyclic alcohol (e.g., cyclobutanol or a cyclopropylmethanol). In yet another instance, if R¹ or R is a branched aliphatic, then the C₄ alcohol can be a C₄-branched alcohol (e.g., 2-butanol, isobutanol, or tert-butanol).

In some instances, when X═OH and Y=—[C(R¹)₂]ₘ—C(R¹)₃, the stabilizer can be a diol. In other instances, when Y=—[C(R¹)₂]ₘ—C(R¹)₃ and at least one R¹═OH or when R═OH, the stabilizer can be a diol. Exemplary diols include, but are not limited to, 1,4-butane diol, propylene-1,3-diol, and the like.

In other instances, when X═Y═OH, the stabilizer can be a triol. In yet other instances, when X═R═OH, the stabilizer can be a triol. In some instances, when at least one of X or Y is —[C(R¹)₂]ₘ—C(R¹)₃ and at least two of R¹ is OH, the stabilizer can be triol. In other instances, when R═OH and X=—[C(R¹)₂]ₘ—C(R¹)₃ and at least two of R¹ is OH, the stabilizer can be triol. Exemplary triols include, but are not limited to, glycerol or derivatives thereof.

In particular embodiments, when R═cycloheteroaliphatic, heterocyclyl, heteroaryl, alkyl-heterocyclyl, alkenyl-heterocyclyl, alkynyl-heterocyclyl, heteroalkyl-heterocyclyl, heteroalkenyl-heterocyclyl, or heteroalkynyl-heterocyclyl, the stabilizer can be a heterocyclyl alcohol (e.g., an optionally substituted heterocyclyl substituted with or more hydroxyls, such as furfuryl alcohol). In other embodiments, when at least one of X or Y is —[C(R¹)₂]M-C(R¹)₃ and at least two of R¹ is cycloheteroaliphatic, heterocyclyl, heteroaryl, alkyl-heterocyclyl, alkenyl-heterocyclyl, alkynyl-heterocyclyl, heteroalkyl-heterocyclyl, heteroalkenyl-heterocyclyl, or heteroalkynyl-heterocyclyl, the stabilizer can be a heterocyclyl alcohol.

In some instances, the alcohol is not a C₁₋₄ alcohol (e.g., methanol, ethanol, propanol, butanol, isopropyl alcohol). In other instances, the alcohol is not a linear C₁₋₄ alkanol. In yet other instances, the alcohol is not a branched C₃ alcohol. In other instances, the alcohol is not a C₂ diol (e.g., ethylene glycol).

### Aldehydes and Ketones

The stabilizer can be an aldehyde having a formula of X—[C(O)]—H, where:

X can be selected from hydrogen, —R¹, —C(R¹)₃ or —[C(R¹)₂]ₘ—C(O)H, wherein each R¹ is independently selected from hydrogen, aliphatic, haloaliphatic, haloheteroaliphatic, heteroaliphatic, aromatic, aliphatic-aromatic, heteroaliphatic-aromatic, or any combinations thereof, and m is an integer from 0 to 10.

The stabilizer can also be a ketone having a formula of X—[C(O)]ₙ—Y, where:

n is an integer from 1 to 2;

each X and Y can be independently selected from —C(R¹)₃, —R², or —[C(R¹)₂]ₘ—C(O)—R², wherein R¹ can be independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof;

R² can be independently selected from aliphatic, haloaliphatic, haloheteroaliphatic, heteroaliphatic, aromatic, aliphatic-aromatic, heteroaliphatic-aromatic, or any combinations thereof;

in which R¹ and R², taken together with the atom to which each are attached, can optionally form a cycloaliphatic or cycloheteroaliphatic, and in which X and Y, taken together with the atom to which each are attached, can optionally form a cycloaliphatic or cycloheteroaliphatic; and

m is an integer from 0 to 10.

In some embodiments, each of R¹ and R² is, independently, alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the aldehyde or ketone may further be substituted with one or more substituents, such as aldehyde (—C(O)H), oxo (═O), alkoxy, amide, amine, hydroxyl, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when X═aromatic, the stabilizer can be an aromatic aldehyde. Exemplary stabilizers include benzaldehyde, 1-naphthaldehyde, phthalaldehyde, and the like.

In other embodiments, when X═aliphatic, the stabilizer can be an aliphatic aldehyde. Exemplary stabilizers include acetaldehyde, propionaldehyde, butyraldehyde, isovalerylaldehyde, and the like.

In yet other embodiments, when X=—[C(R¹)₂]ₘ—C(O)H and m is 0 to 10 or when X═aliphatic or heteroaliphatic substituted with —C(O)H, the stabilizer can be a dialdehyde. Exemplary stabilizers include glyoxal, phthalaldehyde, glutaraldehyde, malondialdehyde, succinaldehyde, and the like.

In some embodiments, when X and Y, taken together with the atom to which each are attached, forms a cycloaliphatic or cycloheteroaliphatic, the stabilizer can be a cyclic ketone. Exemplary cyclic ketones include cyclohexanone, cyclopentanone, and the like.

In other embodiments, when at least one of X or Y=—[C(R¹)₂]ₘ—C(O)—R², the stabilizer can be a diketone. Exemplary diketones include diacetyl, 2,3-pentanedione, 2,3-hexanedione, 3,4-hexanedione, acetylacetone, acetonylacetone, and the like, as well as halogenated forms thereof, such as hexafluoroacetylacetone.

In further embodiments, when at least one of X or Y=—[C(R¹)₂]ₘ—C(O)—R² and X and Y, taken together with the atom to which each are attached, forms a cycloaliphatic or cycloheteroaliphatic, the stabilizer can be a cyclic diketone. Exemplary cyclic diketones include dimedone, 1,3-cyclohexanedione, and the like.

In some instances, when X=—CH₃, the stabilizer can have Y=—C(R¹)₃, in which at least one R¹ is C₂₋₁₀ hydroxyl, aliphatic, haloaliphatic, haloheteroaliphatic, heteroaliphatic, aromatic, aliphatic-aromatic, heteroaliphatic-aromatic, or any combinations thereof. Exemplary stabilizers can include methyl propyl ketone, methyl butyl ketone, hydroxyacetone, and the like.

In other instances, when X=—CH₃, the stabilizer can have Y=—R², in which at least one R² is C₂ alkenyl, C₃₋₁₀ aliphatic, haloaliphatic, haloheteroaliphatic, heteroaliphatic, aromatic, aliphatic-aromatic, heteroaliphatic-aromatic, or any combinations thereof.

Exemplary stabilizers can include methyl vinyl ketone, methyl propyl ketone, methyl butyl ketone, and the like.

In some instances, when X=—CH₂CH₃, the stabilizer can have Y=—C(R¹)₃, in which at least one R¹ is C₂₋₁₀ aliphatic, haloaliphatic, haloheteroaliphatic, heteroaliphatic, aromatic, aliphatic-aromatic, heteroaliphatic-aromatic, or any combinations thereof. Exemplary stabilizers can include methyl propyl ketone, methyl butyl ketone, and the like.

In other instances, when X=—CH₂CH₃, the stabilizer can have Y=—R², in which at least one R² is C₂ alkenyl, C₃₋₁₀ aliphatic, haloaliphatic, haloheteroaliphatic, heteroaliphatic, aromatic, aliphatic-aromatic, heteroaliphatic-aromatic, or any combinations thereof. Exemplary stabilizers can include methyl vinyl ketone, methyl propyl ketone, methyl butyl ketone, and the like.

In yet other instances, when at least one of X or Y=aromatic or aliphatic-aromatic or heteroaliphatic-aromatic, the stabilizer can be an aromatic ketone. Exemplary stabilizers include acetophenone, benzophenone, benzylacetone, 1,3-diphenylacetone, cyclopentyl phenyl ketone, and the like.

In some embodiments, the ketone is not a C₃₋₅ ketone (e.g., not acetone, methyl ethyl ketone, or diethyl ketone). In other embodiments, the ketone does not include X=methyl and Y=methyl or ethyl when i=1. In other embodiments, the ketone does not include X=ethyl and Y=ethyl when n=1.

### Amides

The stabilizer can be an amide having a formula of X—C(O)—NR—[C(O)]ₙ—Y, where: n is 0 or 1;

each X and Y can be independently selected from —R¹ or —[C(R¹)₂]ₘ—C(R¹)₃ or —[C(R¹)₂]ₘ—C(O)—OC(R¹)₃, wherein each R¹ is independently selected from hydrogen, aliphatic, haloaliphatic, haloheteroaliphatic, heteroaliphatic, aromatic, aliphatic-aromatic, heteroaliphatic-aromatic, or any combinations thereof, and m is an integer from 0 to 10; and

each R is independently selected from hydrogen, aliphatic, haloaliphatic, haloheteroaliphatic, heteroaliphatic, aromatic, aliphatic-aromatic, heteroaliphatic-aromatic, or any combinations thereof, in which R and Y, taken together with the atom to which each are attached, can optionally form a cycloheteroaliphatic group, and in which R and X, taken together with the atom to which each are attached, can optionally form a cycloheteroaliphatic group.

In some embodiments, R¹ is alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the amide may further be substituted with one or more substituents, such as aldehyde (—C(O)H), oxo (═O), alkoxy, amide, amine, hydroxyl, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when R and X are taken together to form a cycloheteroaliphatic group, then the stabilizer can be a first cyclic amide. In particular embodiments, when the first cyclic amide includes an unsubstituted 5-membered ring, then Y=—R¹ and each R¹ is independently selected from hydrogen, C₂₋₁₀ aliphatic, C₂₋₁₀ haloaliphatic, C₂₋₁₀ haloheteroaliphatic, C₂₋₁₀ heteroaliphatic, aromatic, aliphatic-aromatic, heteroaliphatic-aromatic, or any combinations thereof. In other embodiments, when the first cyclic amide includes an unsubstituted 5-membered ring, then Y=—[C(Rr)₂]ₘ—C(R¹)₃ and m is an integer from 1 to 10. In some embodiments, the first amide includes substituted 5-membered ring (e.g., in which substitutions can include C₁₋₁₀ aliphatic) and provides a ring-substituted cyclic amide. In yet other embodiments, the first cyclic amide is not N-methyl-2-pyrrolidone (N-methyl-2-pyrrolidinone). Exemplary first cyclic amides include N-n-propyl-2-pyrrolidinone, N-n-butyl-2-pyrrolidinone, N-isobutyl-2-pyrrolidinone, N-t-butyl-2-pyrrolidinone, N-n-pentyl-2-pyrrolidinone, N-(methoxypropyl)-2-pyrrolidinone, N-(methoxybutyl)-2-pyrrolidinone, N-octyl-2-pyrrolidinone, N-cyclohexyl-2-pyrrolidinone, caprolactam, N-methyl-caprolactam, and the like. Exemplary ring-substituted cyclic amides include 1,5-di methylpyrrolidinone, 5-methyl-N-n-propyl-2-pyrrolidinone, 5-methyl-N-n-butyl-2-pyrrolidinone, and the like.

In some embodiments, when R and Y are taken together to form a cycloheteroaliphatic group, the stabilizer can be a second cyclic amide. Exemplary second cyclic amides include N-acetyl pyrrolidine, N-formyl pyrrolidine, N-acetyl piperidine, N-formyl piperidine, N-acetyl morpholine, N-formyl morpholine, N-propionyl morpholine, and the like.

In other embodiments, when X═H and at least one of R or Y═H, then the stabilizer can be a N-alkyl formamide. Exemplary N-alkyl formamides include N-methyl formamide, N-ethyl formamide, N-propyl formamide, and the like.

In yet other embodiments, when X═H and R═Y═C₂₋₁₀ aliphatic, the stabilizer can be an N,N-di-C₂₋₁₀ alkyl formamide. In particular embodiments, the N,N-di-C₂₋₁₀ alkyl formamide is not N,N-dimethyl formamide. Exemplary N,N-di-C₂₋₁₀ alkyl formamides includes N,N-diethyl formamide, N,N-diisopropyl formamide, N,N-dibutyl formamide, and the like.

In some embodiments, when X═H, the stabilizer can be an amide in which at least one of R or Y=hydrogen, C₂₋₁₀ aliphatic, haloaliphatic, haloheteroaliphatic, heteroaliphatic, aromatic, aliphatic-aromatic, heteroaliphatic-aromatic, or any combinations thereof.

In other embodiments, when X═CH₃ and at least one of R or Y═H, the stabilizer can be a N-alkyl acetamide. Exemplary N-alkyl acetamides include N-methyl acetamide, N-ethyl acetamide, N-propyl acetamide, and the like.

In yet other embodiments, when X═CH₃ and R═Y═C₂₋₁₀ aliphatic, the stabilizer can be an N,N-di-C₂₋₁₀ alkyl acetamide. In particular embodiments, the N,N-di-C₂₋₁₀ alkyl acetamide is not N,N-dimethyl acetamide. Exemplary N,N-di-C₂₋₁₀ alkyl acetamides includes N,N-diethyl acetamide, N,N-diisopropyl acetamide, N,N-dibutyl acetamide, and the like.

In some embodiments, when X═—CH₃, the stabilizer can be an amide in which at least one of R or Y=hydrogen, C₂₋₁₀ aliphatic, haloaliphatic, haloheteroaliphatic, heteroaliphatic, aromatic, aliphatic-aromatic, heteroaliphatic-aromatic, or any combinations thereof.

In some embodiments, when X=—[C(R¹)₂]ₘ—C(O)—OC(R¹)₃, the stabilizer can be an ester amide. In particular embodiments, m is an integer from 1 to 10. Exemplary stabilizers include methyl 5-(dimethylamino)-2-methyl-5-oxopentanoate, methyl 5-(dimethylamino)-5-oxopentanoate, methyl 4-(dimethylamino)-2-methyl-4-oxobutanoate, methyl 4-(dimethylamino)-4-oxobutanoate, and the like.

### Amines

The stabilizer can be an amine having a formula of NR¹R²R³, where:

each of R¹, R², and R³ is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof;

in which R¹ and R², taken together with the atom to which each are attached, can optionally form a cycloheteroaliphatic; and

in which R¹, R², and R³, taken together with the atom to which each are attached, can optionally form a cycloheteroaliphatic.

In some embodiments, each of R¹, R², and R³ is independently selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the amine may further be substituted with one or more substituents, such as alkoxy, amide, amine, hydroxyl, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when at least one of R¹, R², and R³ is aliphatic, haloaliphatic, haloheteroaliphatic, or heteroaliphatic, the stabilizer is an alkyl amine. The alkyl amine can include dialkylamines and trialkyl amines. Exemplary alkyl amines include N,N-dimethylisopropylamine, N-ethyldiisopropylamine, trimethylamine, dimethylamine, methylamine, triethylamine, t-butyl amine, and the like.

In other embodiments, when at least one of R¹, R², and R³ includes a hydroxyl, the stabilizer is an alcohol amine. In one instance, at least one of R¹, R², and R³ is an aliphatic group substituted with one or more hydroxyls. Exemplary alcohol amines include 2-(dimethylamino)ethanol, 2-(diethylamino)ethanol, 2-(dipropylamino)ethanol, 2-(dibutylamino)ethanol, N-ethyldiethanolamine, N-tertbutyldiethanolamine, and the like.

In some embodiments, when R¹ and R², taken together with the atom to which each are attached, form a cycloheteroaliphatic, the stabilizer can be a cyclic amine. Exemplary cyclic amines include piperidine, N-alkyl piperidine (e.g., N-methyl piperidine, N-propyl piperidine, etc.), pyrrolidine, N-alkyl pyrrolidine (e.g., N-methyl pyrrolidine, N-propyl pyrrolidine, etc.), morpholine, N-alkyl morpholine (e.g., N-methyl morpholine, N-propyl morpholine, etc.), piperazine, N-alkyl piperazine, N,N-dialkyl piperazine (e.g., 1,4-dimethylpiperazine), and the like.

In other embodiments, when at least one of R¹, R², and R³ includes an aromatic, the stabilizer is an aromatic amine. In some embodiments, at least one of R¹, R², and R³ is aromatic, aliphatic-aromatic, or heteroaliphatic-aromatic. In other embodiments, both R¹ and R² includes an aromatic. In yet other embodiments, R¹ and R² and optionally R³, taken together with the atom to which each are attached, from a cycloheteroaliphatic that is an aromatic. Exemplary aromatic amines include aniline, histamine, pyrrole, pyridine, imidazole, pyrimidine, and the like.

### Carbenes and Derivatives Thereof

The stabilizer can be a carbene having a formula X—(C:)—Y, where:

each of X and Y can be independently selected from H, halo, —[C(R¹)₂]ₘ—C(R¹)₃, —C(O)—R¹, or —C(═NR¹)—R¹, —NR¹R², —OR², —SR², or —C(R²)₃, wherein each of R¹ and R² is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein m is an integer from 0 to 10;

in which R¹ and R², taken together with the atom to which each are attached, can optionally form a cycloheteroaliphatic group, and

in which X and Y, taken together with the atom to which each are attached, can optionally form a cycloaliphatic or cycloheteroaliphatic group.

Furthermore, the stabilizer can be a carbenium cation having a formula R¹—C*(R)—R², wherein each of R, R¹, and R² is independently selected from hydrogen, aliphatic, haloaliphatic, haloheteroaliphatic, heteroaliphatic, aromatic, aliphatic-aromatic, heteroaliphatic-aromatic, or any combinations thereof.

In some embodiments, each R, R¹, and R² independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the carbene may further be substituted with one or more substituents, such as alkoxy, amide, amine, hydroxyl, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof. In any embodiment of a carbene, each of R¹ and R² can be independently selected.

In some embodiments, when at least one of X or Y is halo, the stabilizer can be a halocarbene. Exemplary, non-limiting halocarbenes include dihalocarbene, such as dichlorocarbene, difluorocarbene, and the like.

In some embodiments, when both X═Y=—NR¹R², the stabilizer can be a diaminocarbene. In one instance, each of R¹ and R² is independently aliphatic. Exemplary diaminocarbenes include bis(diisopropylamino) carbene.

In other embodiments, when both at least one of X or Y=—NR¹R² and both R¹ and R² within X or within Y are taken together, with the nitrogen atom to which each are attached, to form a cycloheteroaliphatic group, the stabilizer can be a cyclic diaminocarbene. Exemplary cyclic diamino carbenes include bis(N-piperidyl) carbene, bis(N-pyrrolidinyl) carbene, and the like.

In one instance, when both X═Y=—NR¹R² and an R¹ group from X and an R² group from Y are taken together, with the nitrogen atom to which each are attached, to form a cycloheteroaliphatic group, the stabilizer is an N-heterocyclic carbene. Exemplary N-heterocyclic carbenes include imidazol-2-ylidenes (e.g., 1,3-dimesitylimidazol-2-ylidene, 1,3-dimesityl-4,5-dichloroimidazol-2-ylidene, 1,3-bis(2,6-diisopropylphenyl)imidazol-2-ylidene, 1,3-di-tert-butylimidazol-2-ylidene, etc.), imidazolidin-2-ylidenes (e.g., 1,3-bis(2,6-diisopropylphenyl)imidazolidin-2-ylidene), triazol-5-ylidenes (e.g., 1,3,4-triphenyl-4,5-dihydro-1H-1,2,4-triazol-5-ylidene), and the like.

In some embodiments, when X=—NR¹R² and Y=—SR² and an R¹ group from X and an R² group from Y are taken together, with the nitrogen atom to which each are attached, to form a cycloheteroaliphatic group, the stabilizer is an exemplary cyclic thioalkyl amino carbene. Exemplary cyclic thioalkyl amino carbenes include thiazol-2-ylidenes (e.g., 3-(2,6-diisopropylphenyl)thiazol-2-ylidene and the like).

In some embodiments, when X=—NR¹R² and Y=—C(R²)₃ and an R¹ group from X and an R² group from Y are taken together, with the atom to which each are attached, to form a cycloheteroaliphatic group, the stabilizer is an exemplary cyclic alkyl amino carbene. Exemplary cyclic alkyl amino carbenes include pyrrolidine-2-ylidenes (e.g., 1,3,3,5,5-pentamethyl-pyrrolidin-2-ylidene and the like) and piperidin-2-ylidenes (e.g., 1,3,3,6,6-pentamethyl-piperidin-2-ylidene and the like).

Further exemplary carbenes and derivatives thereof include compounds having a thiazol-2-ylidene moiety, a dihydroimidazol-2-ylidene moiety, an imidazol-2-ylidene moiety, a triazol-5-ylidene moiety, or a cyclopropenylidene moiety. Yet other carbenes and carbene analogs include an aminothiocarbene compound, an aminooxycarbene compound, a diaminocarbene compound, a heteroamino carbene compound, a 1,3-dithiolium carbene compound, a mesoionic carbene compound (e.g., an imidazolin-4-ylidene compound, a 1,2,3-triazolylidene compound, a pyrazolinylidene compound, a tetrazol-5-ylidene compound, an isoxazol-4-ylidene compound, a thiazol-5-ylidene compound, etc.), a cyclic alkyl amino carbene compound, a boranylidene compound, a silylene compound, a stannylene compound, a nitrene compound, a phosphinidene compound, a foiled carbene compound, etc. Further exemplary carbenes include dimethyl imidazol-2-ylidene, 1,3-bis(2,4,6-trimethylphenyl)-4,5-dihydroimidazol-2-ylidene, (phosphanyl)(trifluoromethyl)carbene, bis(diisopropylamino) carbene, bis(diisopropylamino) cyclopropenylidene, 1,3-dimesityl-4,5-dichloroimidazol-2-ylidene, 1,3-diadamantylimidazol-2-ylidene, 1,3,4,5-tetramethylimidazol-2-ylidene, 1,3-dimesitylimidazol-2-ylidene, 1,3-dimesitylimidazol-2-ylidene, 1,3,5-triphenyltriazol-5-ylidene, bis(diisopropylamino) cyclopropenylidene, bis(9-anthryl)carbene, norbornen-7-ylidene, dihydroimidazol-2-ylidene, methylidenecarbene, etc.

### Dienes

The stabilizer can be a diene having a formula of R¹R²—C═C(R¹)—[C(R¹)₂]ₙ—C(R¹)═CR¹R², where:

n is an integer from 0 to 10; and

each of R¹ and R² is independently selected from hydrogen, aliphatic, haloaliphatic, haloheteroaliphatic, heteroaliphatic, aromatic, aliphatic-aromatic, heteroaliphatic-aromatic, or any combinations thereof.

In some embodiments, each R¹ and R² independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the diene may further be substituted with one or more substituents, such as alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In another embodiment, the stabilizer is a diene in the s-cis conformation, which can be provided in linear or cyclic form. Non-limiting dienes include an optionally substituted butadiene, an optionally substituted cyclopentadiene, an optionally substituted o-quinodimethane, and an optionally substituted 1,2-dihydropyridine.

### Esters

The stabilizer can be an ester having a formula of X—[O]ₙ—C(O)—O—Y, where:

n is 0 or 1;

each X and Y can be independently selected from —[C(R¹)₂]ₘ—C(R¹) or —{[C(R¹)₂]ₘ—[O]ₙ}ₚ—C(R¹) or —[C(R¹)₂]ₘ—C(O)—N(R¹)₂ or —[C(R¹)₂]ₘ—C(O)—O—[C(R¹)₂]ₘ—C(R¹), wherein each R¹ is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein m is an integer from 0 to 10 and p is an integer from 1 to 10; and

in which X and Y, taken together with the atom to which each are attached, can optionally form a cycloheteroaliphatic group.

In some embodiments, each R¹ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the ester may further be substituted with one or more substituents, such as alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when X and Y are taken together with the atom to which each are attached in order to form a cycloheteroaliphatic group, the stabilizer can be a cyclic ether. Exemplary cyclic ethers include lactones, such as ε-caprolactone, γ-caprolactone, γ-valerolactone, δ-valerolactone, and the like.

In some embodiments, when at least one of X or Y is —[C(R¹)₂]ₘ—C(O)—N(R¹)₂, then the ester can be an aminoester. Exemplary amino esters include methyl-5-(dimethylamino)-2-methyl-5-oxopentanoate and the like.

In some embodiments, when X=—CH: and n=0, the stabilizer can be an acetate in which Y=—[C(R¹)₂]ₘ—C(R¹) and m is an integer from 2 to 10. In other embodiments, when X═—CH₃ and n=0, the stabilizer can be an acetate in which Y=—[C(R¹)₂]ₘ—C(R¹), m is an integer from 1 to 10, and at least one R¹ is C₁₋₁₀ aliphatic, haloaliphatic, haloheteroaliphatic, heteroaliphatic, aromatic, aliphatic-aromatic, heteroaliphatic-aromatic, or any combinations thereof. In some embodiments, the acetate is not ethyl acetate. Exemplary acetates include n-propyl acetate, isopropyl acetate, n-butyl acetate, t-butyl acetate, iso-butyl acetate, propylene glycol methyl ether acetate, etc., including corresponding acetates of methyl, ethyl, propyl, and butyl mono- and di-ethers of ethylene glycol.

In some embodiments, when at least one of X or Y=—{[C(R¹)₂]ₘ—[O]ₙ}ₚ—C(R¹), the stabilizer can be a glycol based ester. Exemplary glycol based esters include propylene glycol methyl ether acetate, and the like.

In other embodiments, when at least one of X or Y includes a hydroxyl, the stabilizer can be a hydroxy ester. Exemplary hydroxy esters include alpha-hydroxy esters, such as those derived from lactate (e.g., methyl lactate, ethyl lactate, n-propyl lactate, isopropyl lactate, n-butyl lactate, isobutyl lactate, t-butyl lactate, etc.).

In some embodiments, when n=1, the stabilizer can be a carbonate ester. In particular embodiments, X and Y are taken together with the atom to which each are attached in order form a cycloheteroaliphatic group, thereby providing a cyclic carbonate ester. Exemplary carbonate esters include propylene carbonate, diethyl carbonate, glycerol carbonate, and the like.

In other embodiments, when X=—[C(R¹)₂]ₘ—C(O)—O—[C(R¹)₂]ₘ—C(R¹) (and, e.g., n=0), the stabilizer can be a diester. Exemplary diesters include dimethyl 2-methylglutarate, dimethyl succinate, dimethyl adipate, and the like.

### Ethers

The stabilizer can be an ether having a formula of X—O—Y or X—O—[C(R)₂]ₙ—O—Y, where:

n is an integer from 1 to 4;

each X and Y can be independently selected from —[C(R¹)₂]ₘ—C(R¹) or —R² or —[C(R¹)₂]ₚ—O—[C(R¹)₂]ₘ—C(R¹), wherein each of R¹ and R is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein m is an integer from 0 to 10 and p is an integer from 1 to 10;

in which X and Y, taken together with the atom to which each are attached, can optionally form a cycloheteroaliphatic group; and

R² is independently selected from aliphatic, haloaliphatic, haloheteroaliphatic, heteroaliphatic, aromatic, aliphatic-aromatic, heteroaliphatic-aromatic, or any combinations thereof.

In some embodiments, each R and R¹ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the ether may further be substituted with one or more substituents, such as alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In some embodiments, when X and Y are taken together with the atom to which each are attached in order form a cycloheteroaliphatic group, the stabilizer is a cyclic ether. In some embodiments, when in =1 and each R═H, X and Y form a six, seven, eight, nine, or ten-membered ring. In other embodiments, when n=2 and R═H, X and Y form a seven, eight, nine, or ten-membered ring. In yet other embodiments, when n=1 or n=2, then R is aliphatic, haloaliphatic, haloheteroaliphatic, heteroaliphatic, aromatic, aliphatic-aromatic, heteroaliphatic-aromatic, or any combinations thereof. In some embodiments, the ether is not 1,3-dioxolane or 1,4-dioxane. Exemplary cyclic ether includes tetrahydrofuran, 2-methyltetrahydrofuran, 2-methyl-1,3-dioxolane, and the like.

In other embodiments, when at least one of X or Y=aromatic, the stabilizer can be an aromatic ether. Exemplary aromatic ethers include anisole, diphenyl ether, and the like.

In some embodiments, when at least one of X or Y=cycloaliphatic, the stabilizer can be a cycloalkyl ether. Exemplary cycloalkyl ethers include cyclopentyl methyl ether, cyclohexyl methyl ether, and the like.

In other embodiments, when at least one of X or Y=—[C(R¹)₂]ₚ—O—[C(R¹)₂]ₘ—C(R¹), the stabilizer can be a glycol based ether. Exemplary glycol based ethers include diethylene glycol diethyl ether, dipropylene glycol dimethyl ether, poly(ethylene glycol) dimethyl ether, etc., including methyl, ethyl, propyl, and butyl mono- and di-ethers of ethylene glycol, and the like.

### Guanidines

The stabilizer can be a guanidine having a formula of R¹R²N—C(═NR)—NR¹R², where:

each of R, R¹, and R² is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof.

In some embodiments, each of R, R, and R² independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the guanidine may further be substituted with one or more substituents, such as alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

In one embodiment, when both R¹═R²=aliphatic, the stabilizer is an N-tetra-alkyl guanidine. Exemplary N-tetra-alkyl guanidines include N,N,N′,N′-tetramethylguanidine, 2-tert-butyl-1,1,3,3-tetramethylguanidine, and the like.

### Heterocycles

In one embodiment, the stabilizer is a heterocycle having one or more heterocyclyl moieties, as defined herein, such as aromatic heterocycles (e.g., having one or more heteroatoms, such as N, O, and/or S), bicyclic heterocycles (e.g., aromatic bicyclic heterocycles), and the like.

In another embodiment, the heterocycle can include cyclic acids (e.g., having the formula of R—CO₂H, wherein R is optionally substituted heterocyclyl or optionally substituted alkyl-heterocyclyl), cyclic ethers (e.g., having a formula of R¹—O—R², wherein R¹ and R², taken together with the oxygen atom to which each are attached, form an optionally substituted heterocyclyl group, as defined herein, in which the cyclic ether is not 1,3-dioxolane or 1,4-dioxane), cyclic esters (e.g., having a formula of R²—C(O)—OR¹, wherein R¹ and R², taken together with the oxygen atom to which R¹ is attached, form an optionally substituted heterocyclyl group, as defined herein), cyclic carbonate esters (e.g., having a formula of R²O—C(O)—OR¹, wherein R¹ and R², taken together with the oxygen atom to which each are attached, form an optionally substituted heterocyclyl group, as defined herein), and the like.

In another embodiment, the heterocycle can include cyclic amines. An exemplary cyclic amine can have a formula of NR¹R²R³, wherein R¹ and R², taken together with the nitrogen atom to which each are attached, form a heteroaliphatic or heterocyclyl, as defined herein, and wherein R³ is hydrogen, hydroxyl, aliphatic, haloaliphatic, haloheteroaliphatic, heteroaliphatic, aromatic, aliphatic-aromatic, heteroaliphatic-aromatic, or any combinations thereof.

In another embodiment, the heterocycle can include cyclic amides. An exemplary cyclic amide can have a formula of R³—C(O)NR¹R², wherein R¹ and R², taken together with the nitrogen atom to which each are attached, form a heteroaliphatic or heterocyclyl group, as defined herein, and wherein R³ is, independently, hydrogen, hydroxyl, aliphatic, haloaliphatic, haloheteroaliphatic, heteroaliphatic, aromatic, aliphatic-aromatic, heteroaliphatic-aromatic, or any combinations thereof; or wherein R¹ and R³, taken together with the nitrogen atom to which R¹ is attached, form a heteroaliphatic or heterocyclyl group, as defined herein, and wherein R² independently, hydrogen, hydroxyl, aliphatic, haloaliphatic, haloheteroaliphatic, heteroaliphatic, aromatic, aliphatic-aromatic, heteroaliphatic-aromatic, or any combinations thereof; or wherein each of R¹ and R² is, independently, hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein R³ is optionally substituted heterocyclyl or optionally substituted alkyl-heterocyclyl. In some embodiments, the cyclic amide is not N-methyl-2-pyrrolidone.

In another embodiment, the heterocycle can include N-heterocyclic carbenes or cyclic thioalkyl amino carbenes (e.g., as described herein).

Non-limiting heterocycles also include optionally substituted imidazole, optionally substituted triazole, optionally substituted tetrazole, optionally substituted pyrazole, optionally substituted imidazoline, optionally substituted pyrazoline, optionally substituted imidazolidine, optionally substituted pyrazolidine, optionally substituted pyrrole, optionally substituted pyrroline, optionally substituted pyrrolidine, optionally substituted tetrahydrofuran, optionally substituted furan, optionally substituted thiophene, optionally substituted oxazole, optionally substituted isoxazole, optionally substituted isothiazole, optionally substituted thiazole, optionally substituted oxathiolane, optionally substituted oxadiazole, optionally substituted thiadiazole, optionally substituted sulfolane, optionally substituted succinimide, optionally substituted thiazolidinedione, optionally substituted oxazolidone, optionally substituted hydantoin, optionally substituted pyridine, optionally substituted piperidine, optionally substituted pyridazine, optionally substituted piperazine, optionally substituted pyrimidine, optionally substituted pyrazine, optionally substituted triazine, optionally substituted pyran, optionally substituted pyrylium, optionally substituted tetrahydropyran, optionally substituted dioxine, optionally substituted dioxane, optionally substituted dithiane, optionally substituted trithiane, optionally substituted thiopyran, optionally substituted thiane, optionally substituted oxazine, optionally substituted morpholine, optionally substituted thiazine, optionally substituted thiomorpholine, optionally substituted cytosine, optionally substituted thymine, optionally substituted uracil, optionally substituted thiomorpholine dioxide, optionally substituted indene, optionally substituted indoline, optionally substituted indole, optionally substituted isoindole, optionally substituted indolizine, optionally substituted indazole, optionally substituted benzimidazole, optionally substituted azaindole, optionally substituted azaindazole, optionally substituted pyrazolopyrimidine, optionally substituted purine, optionally substituted benzofuran, optionally substituted isobenzofuran, optionally substituted benzothiophene, optionally substituted benzisoxazole, optionally substituted anthranil, optionally substituted benzisothiazole, optionally substituted benzoxazole, optionally substituted benzthiazole, optionally substituted benzthiadiazole, optionally substituted adenine, optionally substituted guanine, optionally substituted tetrahydroquinoline, optionally substituted dihydroquinoline, optionally substituted dihydroisoquinoline, optionally substituted quinoline, optionally substituted isoquinoline, optionally substituted quinolizine, optionally substituted quinoxaline, optionally substituted phthalazine, optionally substituted quinazoline, optionally substituted cinnoline, optionally substituted naphthyridine, optionally substituted pyridopyrimidine, optionally substituted pyridopyrazine, optionally substituted pteridine, optionally substituted benzoxazine, optionally substituted quinolinone, optionally substituted isoquinolinone, optionally substituted carbazole, optionally substituted dibenzofuran, optionally substituted acridine, optionally substituted phenazine, optionally substituted phenoxazine, optionally substituted phenothiazine, optionally substituted phenoxathiine, optionally substituted quinuclidine, optionally substituted azaadamantane, optionally substituted dihydroazepine, optionally substituted azepine, optionally substituted diazepine, optionally substituted oxepane, optionally substituted thiepine, optionally substituted thiazepine, optionally substituted azocane, optionally substituted azocine, optionally substituted thiocane, optionally substituted azonane, optionally substituted azecine, etc. Optional substitutions include any described herein for aryl.

Heterocycles can also include cations and/or salts of any of these. In some embodiments, cationic forms include an optionally substituted alkyl attached to a heteroatom (e.g., N) of a heterocycle. Exemplary cationic forms include thiazolium, as well as salts thereof. Heterocycles can include one or more substituents (e.g., any described herein for aryl or alkyl, such as amine, alkyl, oxo, etc.). Exemplary substituted heterocycles include N-methyl pyrrolidone, N-methylimidazole, 2,6-lutidine, and 4-N,N-dimethylaminopyridine. In some embodiments, the heterocycle includes two or more heteroatoms (e.g., two or more of N, O, and/or S).

### Hydrocarbons

In another embodiment, the stabilizer is a hydrocarbon, including cyclic hydrocarbons (e.g., methylcyclohexane); substituted aromatic hydrocarbons (e.g., halo-substituted benzene, amine-substituted benzene, C₂₋₈ alkyl-substituted benzene, or halo- and alkyl-substituted benzene, such as cumene, aniline, AN-dimethylaniline, etc.): and halocarbons (e.g., a C₂₋₁₂ alkyl having one or more halos). In some instances, the hydrocarbon is not an unsubstituted benzene or a C₁ alkyl-substituted benzene (e.g., toluene, o-xylene, m-xylene, p-xylene). In other instances, the hydrocarbon is not a halo-substituted C₁ hydrocarbon (e.g., chloroform, methylene chloride). In yet other instances, the hydrocarbon is not acetonitrile. In some embodiments, the hydrocarbon is an unsaturated hydrocarbon having one or more double bonds or triple bonds. In other embodiments, the hydrocarbon is an unsaturated, cyclic hydrocarbon (e.g., cyclopentene, cyclohexene, cycloheptene, fluorene, etc.). In particular embodiments, the hydrocarbon is an alkene having one or more double bonds or an alkyne having one or more triple bonds, in which the alkene or the alkyne can be linear or cyclic. Exemplary alkenes include ethene, propene, 1-butene, 1-pentene, 1-hexene, 1-heptene, 1-octene, and 1-nonene, as well as dienes of any of these and positional isomers if available, in which the location of the double bond is changed (e.g., a positional isomer of 1-butene could be 2-butene, etc.). Exemplary alkynes include ethyne, propyne, 1-butyne, 1-pentyne, 1-hexyne, 1-heptyne, 1-octyne, and 1-nonyne, as well as positional isomers if available, in which the location of the triple bond is changed (e.g., a positional isomer of 1-butyne could be 2-butyne, etc.).

### Imines

In one embodiment, the stabilizer is an imine having the formula of R¹N=CR²R³, wherein each of R¹, R², and R³ is, independently, hydrogen, hydroxyl, aliphatic, haloaliphatic, haloheteroaliphatic, heteroaliphatic, aromatic, aliphatic-aromatic, heteroaliphatic-aromatic, or any combinations thereof.

Exemplary imines include Schiff bases having a formula of R¹N═CR²R³, wherein R¹ is hydrogen, hydroxyl, aliphatic, haloaliphatic, haloheteroaliphatic, heteroaliphatic, aromatic, aliphatic-aromatic, heteroaliphatic-aromatic, or any combinations thereof; and each of R² and R³ is, independently, H, hydrogen, hydroxyl, aliphatic, haloaliphatic, haloheteroaliphatic, heteroaliphatic, aromatic, aliphatic-aromatic, heteroaliphatic-aromatic, or any combinations thereof.

In some embodiments, each R¹, R², and R³ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heterocyclyl, heteroaryl, alkyl-aryl, alkenyl-aryl, alkynyl-aryl, alkyl-heterocyclyl, alkenyl-heterocyclyl, alkynyl-heterocyclyl, alkyl-heteroaryl, alkenyl-heteroaryl, alkynyl-heteroaryl, heteroalkyl-aryl, heteroalkenyl-aryl, heteroalkynyl-aryl, heteroalkyl-heterocyclyl, heteroalkenyl-heterocyclyl, heteroalkynyl-heterocyclyl, heteroalkyl-heteroaryl, heteroalkenyl-heteroaryl, heteroalkynyl-heteroaryl, or any combinations thereof. In particular disclosed embodiments, the imine may further be substituted with one or more substituents, such as hydroxyl, alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

### Ionic Liquids

The stabilizer can be an ionic liquid having a cationic moiety and an anionic moiety.

Exemplary cationic moieties include imidazolium (e.g., 1-alkyl-3-methylimidazolium, such as 1-butyl-3-methylimidazolium, 1-ethyl-3-methylimidazolium, etc.), pyridinium (e.g., 1-alkylpyridinium, such as 1-methyl-alkylpyridinium, 1-propyl-alkylpyridinium, 1-butyl-alkylpyridinium, etc.), pyrrolidinium (e.g., N-methyl-N-alkylpyrrolidinium), ammonium (e.g., tetra-alkyl ammonium, such as trioctyl methyl ammonium), phosphonium, thiazolium, triazolium, and the like. Exemplary anionic moieties include tetrafluoroborate (BF₄⁻), hexafluorophosphate (PF₆⁻), bistriflimide ([(CF₃SO₂)₂N]⁻), triflate, acetate, trifluoroacetate, triflimide, halide (e.g., chloride, bromide, iodide), bis(trifluoromethylsulfonyl)imide, methylsulfate, ethyl sulfate, docusate, dicyanamide, and the like.

The cationic moiety may further be substituted with one or more substituents, such as alkyl, alkoxy, amide, amine, thioether, thiol, acyloxy, silyl, cycloaliphatic, aryl, aldehyde, ketone, ester, carboxylic acid, acyl, acyl halide, cyano, halogen, sulfonate, nitro, nitroso, quaternary amine, pyridinyl (or pyridinyl, wherein the nitrogen atom is functionalized with an aliphatic or aryl group), alkyl halide, or any combinations thereof.

### Metal Compounds

In another embodiment, the stabilizer includes metal compounds (e.g., transition metal compounds) and metal salts, such as platinum compounds (e.g., Pt(II)), iron compounds (e.g., Fe(0)), molybdenum compounds (e.g., Mo(0)), chromium compounds (e.g., Cr(0)), titanium compounds (e.g., Ti(IV) or Ti(II)), silver compounds (e.g., Ag(I)), iridium compounds (e.g., Ir(I) or Ir(III)), palladium compounds (e.g., Pd(II)), chromium compounds (e.g., Cr(III)), tantalum compounds (e.g., Ta(V)), cobalt compounds (e.g., Co(II)), copper compounds (e.g., Cu(I)), rhodium compounds (e.g., Rh(I)), osmium compounds (e.g., Os (IV)), and nickel compound (e.g., Ni(I)).

### Onium Compounds

In one embodiment, the stabilizer includes an onium compound, such as a nitronium ion, a nitrosonium ion, a bis(triphenylphosphine)iminium ion, an iminium ion, a diazenium ion, a guanidinium ion, a nitrilium ion, a diazonium ion, a pyridinium ion, a pyrylium ion, a thionitrosyl ion, etc.

### Organosulfur and Organophosphorus Compounds

In one embodiment, the stabilizer includes an organosulfur compound, such as a thioester, a sulfone, a thiosulfinate, a sulfimide, a sulfoximide, a sulfonediimine, an S-nitrosothiol, a thioketone, a thioaldehyde, a thiocarboxylic acid, a thioamide, a sulfonic acid, a sulfinic acid, a sulfenic acid, a sulfonium, an oxosulfonium, or a thiocarbonyl ylide.

In some embodiments, the organosulfur compound is a sulfoxide compound having a formula of X—S(O)—Y, where:

X can be independently selected from hydrogen or —[C(R¹)₂]ₘ—C(R¹); and

Y can be independently selected from hydrogen or —[C(R¹)₂]ₙ—C(R¹), wherein each R¹ is independently selected from hydrogen, hydroxyl, aliphatic, haloaliphatic, haloheteroaliphatic, heteroaliphatic, aromatic, aliphatic-aromatic, heteroaliphatic-aromatic, or any combinations thereof, and wherein m is an integer from 1 to 10 and n is an integer from 0 to 10. In particular embodiments, the sulfoxide is not dimethyl sulfoxide.

In yet another embodiment, the stabilizer includes an organophosphorus compound, such as a phosphate ester, a phosphate amide, a phosphonic acid, a phosphinic acid, a phosphonate a phosphinate, a phosphine oxide, a phosphine imide, or a phosphonium salt.

Exemplary organophosphorus compounds include phosphoric acid and trialkylphosphate.

### Other Stabilizer

Various stabilizers are provided in Table 1 below.

TABLE 1

| | | Vapor pressure | |
|---|---|---|---|
| Class | Name | [Torr, at 25° C.] | Structure |
| Acid | lactic acid | 0.0813 | |
| Acid | propionic acid | 3.53 | |
| Acid | citric acid | 0.000000017 | |
| Acid | malic acid | 0.000000033 | |
| Acid | benzoic acid | 0.0007 | Aryl acid |
| Acid | niacin | 0.000094 | Aryl acid |
| Alcohol | 1-dodecanol | 0.000848 | |
| Alcohol | 1-undecanol | 0.00297 | |
| Alcohol | dianhydro-d-glucitol (isosorbide) | 0.0003 | Cyclic ether/alcohol |
| Alcohol | 1,2-butanediol | 0.05 | Diol |
| Alcohol | 1,2-hexanediol | 0.0179 | Diol |
| Alcohol | 1,2-propanediol | 0.13 | Diol |
| Alcohol | 1,3-butanediol | 0.02 | Diol |
| Alcohol | 1,3-propanediol | 0.0441 | Diol |
| Alcohol | 1,5-pentanediol | 0.0039 | Diol |
| Alcohol | 2,2,4-trimethyl-1,3-pentanediol | 0.0171 | Diol |
| Alcohol | 2-methyl-2,4-pentanediol | 0.07 | Diol |
| Alcohol | polypropylene glycol | 0.0105 | Diol |
| Alcohol | propylene glycol | 0.13 | Diol |
| Alcohol | tetraethylene glycol | 0.0000465 | Diol |
| Alcohol | (2-(2-methoxymethylethoxy) methylethoxy) propanol | 0.02 | Ether alcohol |
| Alcohol | 1-(2-methoxy-1-methylethoxy)-2-propanol | 0.1 | Ether alcohol |
| Alcohol | 1-ethoxy-2-propanol | 7.5 | Ether alcohol |
| Alcohol | 1-propoxy-2-propanol | 2.21 | Ether alcohol |
| Alcohol | 2,2-dimethyl-1,3-dioxolane-4-methanol | 0.2 | Ether alcohol |
| Alcohol | 3-methyl-3-methoxybutanol | 0.7 | Ether alcohol |
| Alcohol | diethylene glycol hexyl ether | 0.000503 | Ether alcohol |
| Alcohol | diethylene glycol mono-n-butyl ether | 0.0219 | Ether alcohol |
| Alcohol | dipropylene glycol methyl ether | 0.5 | Ether alcohol |
| Alcohol | dipropylene glycol monobutyl ether | 0.002 | Ether alcohol |
| Alcohol | dipropylene glycol propyl ether | 0.035 | Ether alcohol |
| Alcohol | glycofurol | 0.002 | Ether alcohol |
| Alcohol | polyethylene glycol monomethyl ether | 0.05 | Ether alcohol |
| Alcohol | propylene glycol n-butyl ether | 1.4 | Ether alcohol |
| Alcohol | 1,1′-dimethyldiethylene glycol | 0.00628 | Ether diol |
| Alcohol | tripropylene glycol | 0.000482 | Ether diol |
| Alcohol | tripropylene glycol n-butyl ether | 0.01 | Ether diol |
| Alcohol | 1-methoxy-2-propanol | 12.5 | Ether/alcohol |
| Alcohol | glycerol | 0.000168 | triol |
| Aldehyde | acetaldehyde | 902 | |
| Amide | niacinamide | 0.00042 | |
| Amide | 1-butyl-2-pyrrolidinone | 0.098 | Pyrrolinone |
| Amine | histamine | — | Aryl amine |
| Ester | 1-methoxy-2-propyl acetate | 3.92 | |
| Ester | dimethyl ethylsuccinate | 0.3 | |
| Ester | dimethyl glutarate | 0.178 | |
| Eister | dimethyl succinate | 0.432 | |
| Ester | ethyl butyrate | 14 | |
| Ester | ethyl formate | 245 | |
| Ester | isopropyl myristate | 0.0000935 | |
| Ester | methyl 9-decenoate | 0.1 | |
| Ester | methyl dodec-9-enoate | 0 | |
| Ester | methyl laurate | 0.00411 | |
| Ester | methyl oleate | 0.00000629 | |
| Ester | propyl acetate | 35.9 | |
| Ester | γ-valerolactone | 0.24 | |
| Ester | butyl 3-hydroxybutanoate | 0.0147 | Alcohol ester |
| Ester | butyl-2-hydroxy-2-methylbutyrate | 0.51 | Alcohol ester |
| Ester | ethyl 3-hydroxybutyrate | 0.4 | Alcohol ester |
| Ester | ethyl lactate | 3.75 | Alcohol ester |
| Ester | isopropyl 3-hydroxybutyrate | 0.0984 | Alcohol ester |
| Ester | triethyl citrate | 0.000687 | Alcohol ester |
| Ester | trimethyl hydroxypentyl isobutyrate | 0.004 | Alcohol ester |
| Ester | dimethylcarbonate | 55.364 | Carbonate ester |
| Ester | ethylene carbonate | 0.0098 | Carbonate ester |
| Ester | propylene carbonate | 0.045 | Carbonate ester |
| Ester | propylene carbonate | 0.045 | Carbonate ester |
| Ester | 4-ethyl-1,3-dioxolan-2-one | 0.000947 | Cyclic carbonate ester |
| Ester | 4-hydroxymethyl-1,3-dioxolan-2-one | 0.00000363 | Cyclic carbonate ester |
| Ester | dipropylene glycol methyl ether acetate | 0.13 | Ether ester |
| Ester | propylene glycol methyl ether acetate | 3.92 | Ether ester |
| Ester | 3-methoxybutyl acetate | 1.2 | Ether ester |
| Ester | ethyl 3-ethoxypropionate | 1.8 | Ether ester |
| Ether | cyclopentyl methyl ether | 44.9 | |
| Ether | anisole | 3.54 | Aryl ether |
| Ether | dimethyl isosorbide | 0.073 | Cyclic ether |
| Ketone | methyl isobutyl ketone | 19.9 | |
| Ketone | diacetyl | 56.8 | Diketone |

Any of the stabilizers described herein include unsubstituted and/or substituted forms of the compound. Non-limiting exemplary substituents include, e.g., one, two, three, four, or more substituents independently selected from the group consisting of: (1) C₁₋₆ alkoxy (e.g., —O—R, in which R is C₁₋₆ alkyl); (2) C₁₋₆ alkylsulfinyl (e.g., —S(O)—R, in which R is C₁₋₆ alkyl); (3) C₁₋₆ alkylsulfonyl (e.g., —SO₂—R, in which R is C₁₋₆ alkyl); (4) amine (e.g., —C(O)NR¹R² or —NHCOR¹, where each of R¹ and R² is, independently, selected from hydrogen, aliphatic, heteroaliphatic, haloaliphatic, haloheteroaliphatic, aromatic, as defined herein, or any combination thereof; or R¹ and R², taken together with the nitrogen atom to which each are attached, can form a heterocyclyl group, as defined herein); (5) aryl; (6) arylalkoxy (e.g., —O-L-R, in which L is alkyl and R is aryl); (7) aryloyl (e.g., —C(O)—R, in which R is aryl); (8) azido (e.g., —N₃); (9) cyano (e.g., —CN); (10) aldehyde (e.g., —C(O)H); (11) C₃₋₈ cycloalkyl; (12) halo; (13) heterocyclyl (e.g., as defined herein, such as a 5-, 6- or 7-membered ring containing one, two, three, or four non-carbon heteroatoms); (14) heterocyclyloxy (e.g., —O—R, in which R is heterocyclyl, as defined herein); (15) heterocyclyloyl (e.g., —C(O)—R, in which R is heterocyclyl, as defined herein); (16) hydroxyl (e.g., —OH); (17) N-protected amino; (18) nitro (e.g., —NO₂); (19) oxo (e.g., =O); (20) C₁₋₆ thioalkoxy (e.g., —S—R, in which R is C₁₋₆ alkyl); (21) thiol (e.g., —SH); (22) —CO₂R¹, where R¹ is selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (23) —C(O)NR¹R², where each of R¹ and R² is, independently, selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); (24) —SO₂R¹, where R¹ is selected from the group consisting of (a) C₁₋₆ alkyl, (b) C₄₋₁₈ aryl, and (c) C₁₋₆ alkyl-C₄₋₁₈ aryl (e.g., -L-R, in which L is C₁₋₆ alkyl and R is C₄₋₁₈ aryl); (25) —SO₂NR¹R², where each of R¹ and R² is, independently, selected from the group consisting of (a) hydrogen, (b) C₁₋₆ alkyl, (c) C₄₋₁₈ aryl, and (d) C₁₋₆ alkyl-C₄₋₁₈ aryl (e.g., -L-R, in which L is C₁₋₆ alkyl and R is C₄₋₁₈ aryl); and (26) —NR¹R², where each of R¹ and R² is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alkyl-C₄₋₁₈ aryl (e.g., -L-R, in which L is C₁₋₆ alkyl and R is C₄₋₁₈ aryl), (h) C₃₋₈ cycloalkyl, and (i) C₁₋₆ alkyl-C₃₋₈ cycloalkyl (e.g., -L-R, in which L is C₁₋₆ alkyl and R is C₃₋₈ cycloalkyl), wherein in one embodiment no two groups are bound to the nitrogen atom through a carbonyl group or a sulfonyl group. In particular embodiments, the stabilizer is not acetonitrile.

### Deposition Process

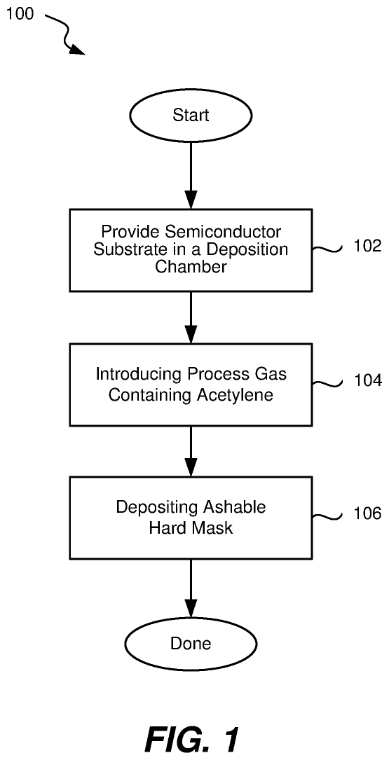

FIG. 1 illustrates stages in a general process flow for forming an ashable hard mask in accordance with certain embodiments. Ashable hardmasks are carbon-based films used as etch masks that can be removed after use by oxidation. In certain embodiments, they are amorphous carbon-based films. Amorphous carbon-based films may also be used to form other types of films used in semiconductor processing. In the depicted embodiment, a method 100 begins with providing a semiconductor substrate in a deposition chamber (block 102). For example, a semiconductor substrate may be a 300 mm semiconductor wafer and the deposition chamber may be a Lam Research Vector® module. A precursor process gas including acetylene is then introduced into the chamber (block 104). Depending on deposition chamber size and other process parameters, the flow rate of acetylene may be about 3,000-10,000 standard cubic centimeters per minute (sccm) during the deposition process. In one embodiment, the flow rate of acetylene may be about 5,000-8,000 sccm. The process gas may also include other carbon containing precursors, such as methane, ethylene, propylene, butane, cyclohexane, benzene and toluene, and the like.

A carrier gas may be used to dilute the precursor. The carrier gas may include any suitable carrier gas employed in semiconductor processing, such as helium, argon, nitrogen, hydrogen, or a combination of these. The overall carrier gas flow rate may depend on deposition chamber size and other process parameters and may range from about 500-10,000 sccm. In a specific embodiment, nitrogen and helium are used as carrier gases having corresponding flow rates ranges of about 500-5,000 sccm and about 300-3,000 sccm. Other stages of semiconductor processing may include different processing gases and different flow rates. For example, chamber cleaning may not involve carbon containing precursors.

In the depicted embodiment, an ashable hard mask is then deposited on the semiconductor substrate by a plasma enhanced chemical vapor deposition (PECVD) or other deposition processes (block 106).

### Methods and Apparatus for Providing Acetylene

Acetylene can begin to decompose at pressures above 15 psig. Given acetylene's propensity to violently decompose, acetylene cylinders are supplied with safety pressure regulators that limit the pressure in receiving lines to 15 psig. However, the overhead pressure inside the cylinder may exceed 200 psi when used with safety devices. Considering that deposition chambers are usually operated at low pressures, 15 psig or less is a sufficiently high driving pressure to flow an acetylene gas stream through a pre-process module and into a deposition chamber. As an example, a deposition process may employ a flow rate of about 6750 sccm (6.75 L/min) while the deposition chamber is maintained at about 8 Torr. As discussed above, flow rates and chamber pressures may vary depending on process requirements.

In certain embodiments, the acetylene source is a cylinder that contains a filler material and stabilizer in addition to the safety relief devices. In certain embodiments, standard-sized hollow steel cylinders that are conventionally used for compressed gas service are used for acetylene. In certain embodiments, ganged cylinders, i.e., cylinders connected to a common gas line.

While the acetylene source may be a bottle or cylinder, it may also be a tank or a facility wide supply of acetylene (e.g., an acetylene resource plumbed into the facility).

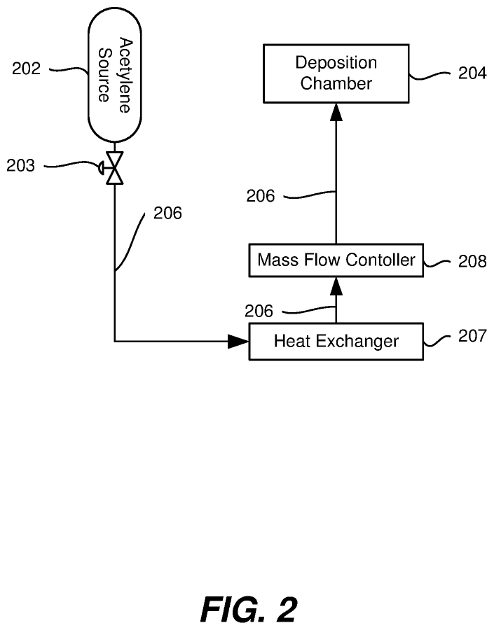

FIG. 2 is a block diagram depicting an example deposition system employing an acetylene source 202 and a deposition chamber 204, as well as other components. The acetylene canister or other acetylene source 202 has a corresponding pressure regulator 203 and is connected to the deposition chamber. The acetylene source contains acetylene and a stabilizer. In certain embodiments, the acetylene source is pressured to over about 200 psi.

When the acetylene gas stream is discharged from the acetylene source, it first passes through the pressure regulator 203 where the service pressure of the cylinder is reduced to a safe level below 15 psig. The acetylene is then passed via a processing line 206 and into the deposition chamber 204. Suitable materials for the processing line 206 include steel and wrought iron. Generally, cast iron, unalloyed copper, silver, or mercury are avoided because of possibility of forming explosive acetylides. The acetylene gas stream in the processing line may include some stabilizer vapor in addition to acetylene. The stabilizer concentration in the processing line may be in range of about 0.01% to 0.1% depending on the current service pressure of the acetylene cylinder. The temperature of the acetylene gas stream in the line depends on the storage conditions of the acetylene cylinder, evaporation rate and other factors. In certain embodiments, the stream may first be passed through a heat exchanger 207 to regulate the temperature of the acetylene during deposition. In certain embodiments, the heat exchanger 207 can maintain the temperature of the acetylene gas stream between about 10° C. and 50° C. In some cases, the temperature is maintained between about 15° C. and 30° C.

The acetylene gas stream then flows through the mass flow controller (MFC) 208 and into the deposition chamber 204. The MFC 208 may be calibrated relative to specific ranges for the properties of the reduced stabilizer concentration acetylene gas stream, such as temperature, composition, pressure, desired flow rate, and others. The MFC 208 may be fitted with a closed loop control system which is given an input signal by the operator or an external system, wherein the input value is compared to a value from the mass flow sensor and a valve of the MFC is adjusted accordingly to achieve the required flow rate.

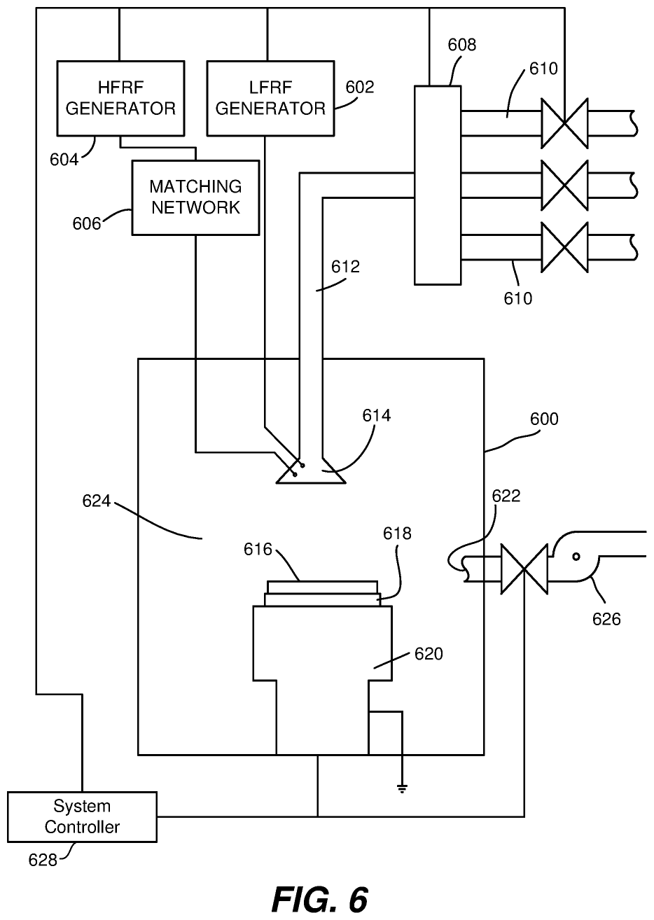

Finally, the acetylene gas stream flows into the deposition chamber 204. An example of the deposition chamber 204 is described in more details in the context of FIG. 6. The acetylene gas stream may be used as a carbon containing precursor that is used to form a layer of high carbon content material on a semiconductor substrate during fabrication of an electronic device. This process is performed in the deposition chamber 204.

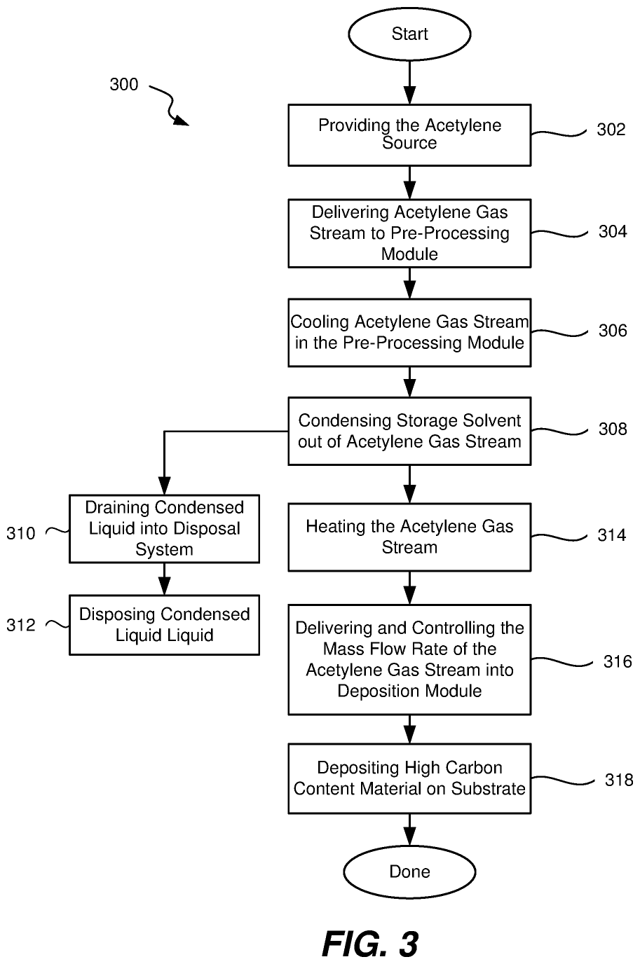

FIG. 3 is a flowchart illustrating some stages in a process flow for treating an acetylene gas stream in accordance with certain embodiments. The process starts with providing an acetylene source 302. By way of example, acetylene for this example may be supplied in cylinders (also referred to as bottles) storing 200-500 cubic feet of acetylene (at standard temperature and pressure) where acetylene is dissolved in acetone. Acetylene is dissolved in a stabilizer and may be contained in a metal cylinder with porous material, such as agamassan.

Returning to FIG. 3, the next operation involves delivering acetylene from the acetylene source to the pre-processing module. The delivery of the acetylene into the pre-processing module 304 is driven by the pressure differential within the overall system and may be controlled by a valve on acetylene source and a mass flow controller between the pre-processing module and the deposition chamber. In certain embodiments, the pressure drop within the pre-processing module is not substantial and depends on lengths and effective diameters all paths that acetylene gas stream takes. Additionally, the pressure drop may be affected by the temperature and the composition of the acetylene gas stream. Once the acetylene gas stream fills the pre-processing module after opening the valve on the acetylene source, the acetylene gas stream may experience at least two flow regimes. One is when the deposition process operation does not require any acetylene, for example during the deposition chamber cleaning, and the acetylene gas stream remains stationary inside the pre-processing module. Another regime is when the acetylene gas stream flows through the pre-processing module and into the deposition chamber.

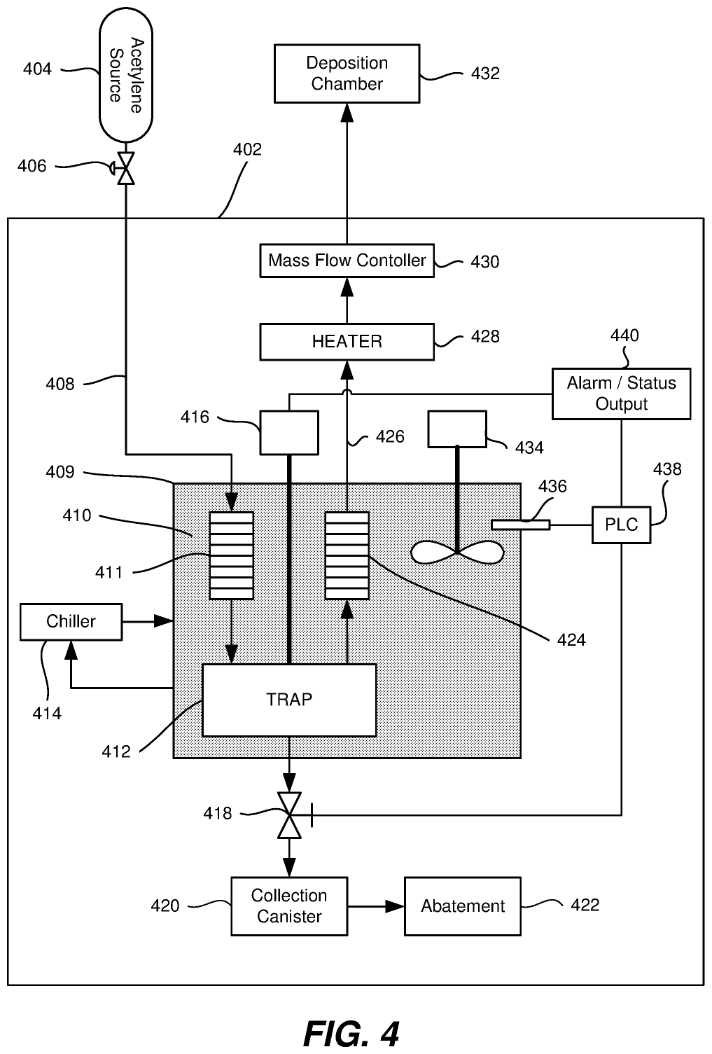

As the acetylene gas stream flows through the pre-processing module it is cooled to a certain temperature (block 306). This facilitates condensation and removal of the acetylene stabilizer. The pre-processing module may include a variety of means to achieve the requisite cooling. In a specific embodiment, the acetylene gas stream passes through a heat exchanger that is maintained in contact with a cooler material. A variety of heat exchanger types may be used for cooling, for example a shell and tube heat exchanger, a plate heat exchanger, a regenerative heat exchanger, an adiabatic wheel heat exchanger, and others. In a specific embodiment a set of two spiral heat exchangers is used. Additional details of an example pre-processing module are described below in the context of FIG. 4.

The heat exchanger may be submerged into a bath containing coolant. As an example, the heat exchangers are submerged in ethylene glycol maintained at temperatures of −30° C. to −60° C. The design of the heat exchangers and the flow rate of the acetylene gas stream may be such that the temperature of the stream leaving the heat exchangers is within a few degrees from the temperature of the coolant. When entering the pre-process module the acetylene gas stream may contain between about 0.5% to 5% of the stabilizer vapor. Lowering the temperature of the acetylene gas stream can reduce the stabilizer vapor in the acetylene gas stream by condensation. The concentration of stabilizer vapor remaining in the gaseous acetylene (after condensation of stabilizer vapor) depends on the temperature of the acetylene gas stream in the heat exchangers, the initial concentration of the stabilizer in the stream, the flow rate of the stream, and other process parameters. While lower temperatures may remove much of the stabilizer from the stream, too low a temperature may cause more acetylene to dissolve in the condensed stabilizer. Therefore, the temperature of the exiting acetylene gas stream may be based on desired final concentrations of stabilizer, losses of acetylene, and overall pre-processing module design.

The condensation of the stabilizer typically occurs on the inside walls of the heat exchangers (see block 308). The surface area of the walls of the heat exchangers is sufficiently large to provide adequate heat transfer and condensation. The condensed stabilizer progresses gravitationally and by gas pressure through the heat exchangers and to the bottom of the liquid trap, where it is temporary collected before being drained into a disposal system. The acetylene gas stream also passes through the trap while carrying some liquid stabilizer droplets in the mist form that may be removed in a mist barrier.

When the liquid level at the bottom of the trap reaches or exceeds the set maximum level, the level sensor ensures that the liquid is drained into the disposal system (see block 310). In certain embodiments, the level sensor sends a signal to the control system that opens a draining valve of the disposal system. The liquid is then gravitationally drained into a collection canister, which is maintained at low overhead gas pressures for safety reasons. The condensed stabilizer may contain substantial amount of highly soluble acetylene. Some of this acetylene may be evaporated from the stabilizer, which may be vented into an abatement unit. The stabilizer may then be disposed (see block 312). A variety of methods may be used for abatement of the stabilizer. Alternatively, the liquid may be destroyed by incineration.

The purified acetylene gas stream is then passed through the heater to increase the temperature of the stream to a level suitable for use in the deposition process (see block 314).

The temperature of the stream leaving the heat-exchangers and trap portions of pre-processing module may be close to the temperature of the coolant. In one embodiment, the heat-exchange fluid may be kept at about −30° C. to −60° C. In one embodiment, the gas stream of acetylene and remaining stabilizer is heated to about between about 10° C. and 40° C. Additionally, the heater may be designed to avoid overheating of the acetylene gas stream, especially when the stream is stagnant in the heater and the deposition process operation does not require any acetylene.

Returning to FIG. 3, the gas stream then flows through the mass flow controller and into the deposition chamber (see block 316). A deposition process requires delivery of the acetylene gas stream at controlled flow rates and only during the certain operations, such as ashable mask pre-coat and ashable mask deposition. The delivery rate and timing may be controlled using a mass flow controller.

Finally, the acetylene gas stream is delivered into the deposition chamber where the high carbon content material is deposited on the substrate (see block 318). In general, a high carbon content material is a material containing at least about twenty-five atomic percent carbon and frequently at least about fifty atomic percent carbon. For diamond-like and graphitic films, carbon may account for up to about 100 atomic percent of the films.

In one embodiment, a process for depositing the ashable hard mask may include the following operations: undercoat deposition, ashable hard mask pre-coat, ashable hard mask deposition, chamber cleaning at high pressure, and chamber cleaning at low pressure. The acetylene gas mass flow controller is shut during the remaining operations not involving the pre-coat or ashable hard mask deposition, which may be a significant part of the overall process. However, the valve from the acetylene source may remain open during this period and the acetylene gas stream remains in the pre-process module pressurized by the acetylene source.

FIG. 4 presents a simple block diagram depicting a pre-processing module 402 and other related apparatuses, such as an acetylene source 404 with a corresponding pressure regulator 406 and a deposition chamber 432. The acetylene source 404 contains acetylene and a stabilizer (e.g. any stabilizer(s) described herein). In certain embodiments, the acetylene source 404 is pressured to over 200 psi. The acetylene source may be a tank or a facility wide supply of acetylene (e.g., an acetylene resource plumbed into the facility). In some embodiments, the acetylene source 404 is an acetylene cylinder, such as described above, that contains a filler material and the safety relief devices.

When the acetylene gas stream is discharged from the acetylene source 404, it first passes through the pressure regulator 406 where the service pressure of the cylinder is reduced to a safe level below 15 psig. The acetylene is then passed through a processing line 408 and into the pre-processing module 402. In some embodiments, the pre-processing module 402 includes a liquid bath 409, and the processing line 408 serves as an inlet for the acetylene gas stream into the bath 409. The acetylene gas stream in the processing line 408 may include some stabilizer vapor in addition to acetylene. As an example, the stabilizer concentration in the processing line is typically in range of about 0.5% to 5% depending on the current service pressure of the acetylene cylinder. The temperature of the acetylene gas stream in the line 408 depends on the storage conditions of the acetylene cylinder, evaporation rate and other factors.

In one embodiment, the liquid bath 409 may contain a coolant 410. For example, the coolant may comprise ethylene glycol and water, but other coolants may also be used. For example, Dynalene HF-LO (aliphatic hydrocarbon blend), Dynalene MV (hydrocarbon blend), and/or Syltherm™ XLT (polydimethylsiloxane liquid) may be used. One or more heat exchangers in the acetylene flow path may also be provided in liquid bath 409. For example, in the depicted embodiment, one heat exchanger 411 is attached to the processing line 408, while another heat exchanger 424 is attached to an exit line 426. However, the bath may contain any number of heat exchangers. The number and the design of the heat exchanger depends on the flow rates in the processing line 408, the required concentration of the stabilizer in the exit line 426, various design parameters of the liquid bath 409, and other factors. The liquid bath 410 may include a level sensor 436 that sends a signal to a programmable logic controller (PLC) 438.

The acetylene gas stream is initially cooled in the heat exchanger 411. Depending the process requirements, the acetylene gas stream may be cooled to temperature within a few degrees of that of the coolant. Various heat exchanger types may be used. In some cases, a coiled heat exchange design is used with stainless steel (SS) tubing, such as 316 SS, of about 0.5″ diameter and the surface area of between about 100 to 1000 square inches. In one specific embodiment, the surface area of the heat exchangers is between about 200 and 600 square inches. Initial condensation of the stabilizer from the acetylene gas stream occurs on the wall of the heat exchanger 411. Many stabilizers have a substantially higher boiling temperature than acetylene. However, some acetylene may dissolve in the liquid stabilizer that is present throughout the pre-processing module 402. While it is desirable to cool the acetylene gas stream to very low temperatures to remove most of the acetone or other stabilizer, a minimal temperature may exist to minimize acetylene losses into the liquid stream. In one embodiment, the acetylene gas stream is cooled down to −30° C. to −60° C. inside the pro-processing module 402.

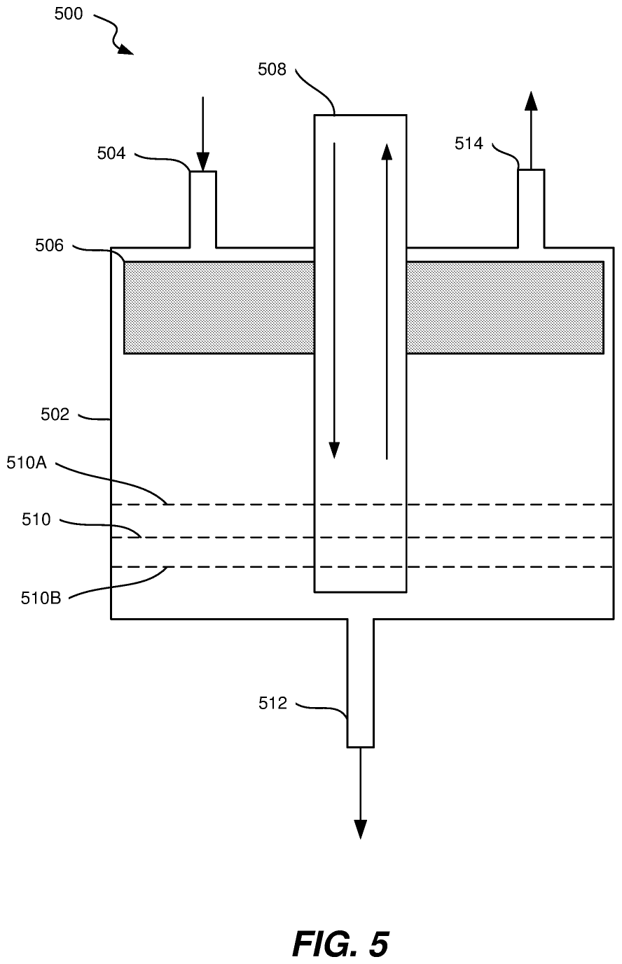

The condensed stabilizer and the acetylene gas stream proceed from heat exchanger 411 to a liquid trap 412. The flow of condensed stabilizer is driven by gravity and the concurrent flow of the gas stream based on the pressure differential with the overall system. The trap 412 is designed to separate the condensed stabilizer from the acetylene gas stream and collect the condensed stabilizer at the bottom of the trap. The liquid collected at the bottom of the trap 412 is primarily condensed stabilizer but may also include some dissolved acetylene. The liquid serves as a barrier for the acetylene gas stream and prevents it from escaping into a collection canister 420. The collected liquid is allowed to escape into the exit line 426. Therefore, the liquid level may be maintained between certain minimum and maximum levels within the trap. In certain embodiments, a level sensor 416 is employed to maintain the liquid level. Alternatively, a simple mechanical liquid trap may be used in a line leading to the collection canister 420. For example, a simple U-, S-, or J-shaped pipe trap may be installed in this line. In some embodiments, a radar sensor, suitable for hazardous environment, using either wavelength in Infrared Red and Radio Frequency regions may be used. In one specific embodiment, a sensor operating at 6.3 GHz is used to track the level of the fluid. The level sensor 416 then sends a signal to a programmable logic controller (PLC) 438. Such signals can also provide an alarm or a status output 440. More details on the operations of an example trap are described in the context of the FIG. 5.

To reach and maintain desired temperature of the bath 409, the coolant 410 is circulated through a chiller 414. Any type of chiller may be used. In certain embodiments, the chiller 414 uses a cyclic refrigeration principle, such as a reverse—Rankine vapor-compression refrigeration. The chiller 414 may be located in a separate facility and includes a pump to circulate the coolant 410 between the bath 409 and the chiller 414. The bath may also include an agitator 434 that provides additional forced convection of the coolant in the bath 409. The agitator 434 may include a motor and a propeller-type mixer at the end of the shaft of the motor that extends into the bath. The motor may be of any type, such as electrical or pneumatic. The agitator 434 may be positioned close to the heat exchangers 411 and 424 to ensure adequate coolant flow around the external surfaces of the heat exchangers.

The gas stream from the trap 412 may be directed into another heat exchanger 424 that is similarly submerged into the coolant 410 of the bath 409. Whether another heat exchanger 424 is employed may depend on the temperature of the coolant, flow rates of the acetylene gas stream, and design of all heat exchangers present in the pre-processing module 402. In certain embodiments, the heat exchanger 424 is approximately thermally equivalent to the first heat exchanger 411. The heat exchanger 424 is installed after the trap 412 and before the exit line 426, with respect to the flow of the acetylene gas stream. The heat exchanger 424 provides for additional cooling of the acetylene gas stream and further condensation of the stabilizer from the stream. The condensed liquid is drained inside the heat exchanger 424 back into the trap by gravitation and against the flow of the acetylene gas stream. Therefore, the internal size of piping used in certain embodiments may be sufficient to accommodate for this reverse flow. Some of the condensed liquid may be present as a mist in the gas stream. A mist trap may be integrated along the flow of the acetylene gas stream in or before the exit line 426.

The liquid being collected from one or more heat exchangers then accumulates in the trap. When it reaches a certain level, a drain valve 418 leading to the collection canister 420 opens and the liquid gravitationally flows into the collection canister 420. The valve closes when the liquid level reaches or falls below a certain minimum liquid level also controlled by the level sensor 416. The collection canister 420 is kept at a low temperature and low pressure to avoid reaching pressures above 15 psig. The condensed stabilizer may contain a substantial amount of condensed and dissolved acetylene. The temperature of the liquid leaving the bath 409 is close to the temperature of the bath 409 itself. In certain embodiments, the bath 409 temperature is maintained at about −30° C. to −60° C. Increasing the temperature of the liquid in the collection canister 420 will lead to acetylene evaporation from the liquid. To prevent the acetylene pressure from exceeding 15 psig, the collection canister 420 is maintained and near atmospheric pressure. The liquid is then transferred or evaporated into an abatement unit 422. Various method of disposing the liquid may be used. In some cases, the abatement unit 422 burns anything supplied from the collection canister 420.

The acetylene gas stream then proceeds into the exit line 426. The concentration of the stabilizer is substantially reduced in the acetylene gas stream at this point. The temperature of this stream in the exit line 426 may be within a few degrees of the coolant temperature. Since many of gas properties are dependent on the temperature and may affect operation of the mass flow controller 430 and impact the deposition process in the deposition chamber 432, the stream is first passed through a heater 428. Various heater types may be used. In certain embodiments, the heater can maintain the temperature of the acetylene gas stream between about 10° C. and 50° C. In one specific embodiment, the temperature is maintained between about 15° C. and 30° C.

The acetylene gas stream with reduced stabilizer concentration then flows through the mass flow controller (MFC) 430 and into the deposition chamber 432. The MFC 430 may be calibrated relative to specific ranges for the properties of the acetylene gas stream, such as temperature, composition, pressure, desired flow rate, and others. The MFC 430 may be fitted with a closed loop control system which is given an input signal by the operator or an external system, wherein the input value is compared to a value from the mass flow sensor and a valve of the MFC is adjusted accordingly to achieve the required flow rate.

Finally, the acetylene gas stream flows into the deposition chamber 432. The pre-processing module may be designed to interface with the deposition chamber. This may involve providing it with specifically designed and/or selected flow tubes (including specific sizes, geometries and orientations) at the interface as well as specific fittings for direct coupling to the deposition chamber. Several types of fittings may be used for connecting the deposition chamber 432 to the pre-processing module 402. For example, Swagelok VCR Face-Seal fitting or Swagelok VCR tube fittings may be used for this interconnection. Other vacuum types and low-pressure gas connection types of fittings may be used. In some embodiments, the fittings may be specifically designed be compatible with semiconductor equipment and overall semiconductor processing. An example of the deposition chamber 432 is described in more details in the context of FIG. 6. As mentioned, the acetylene gas stream may be used as a carbon containing precursor that is used to form a layer of high carbon content material on a semiconductor substrate during fabrication of an electronic device. This process is performed in the deposition chamber 432.

FIG. 5 is a schematic diagram of a trap 500 used in an acetylene pre-processing module such as disclosed herein. With reference to FIG. 5, trap 500 may be an example of the element 412. The trap 500 includes a body 502 which includes a gas stream inlet line 504, a gas stream outlet line 504, and a condensed liquid outlet 512. The gas stream inlet line 504 and the gas stream outlet line 504 may be attached to heat exchangers or other elements of the pre-processing module. The acetylene gas stream enters into the trap 500 through the inlet line 504. The stream has been already cooled before entering the trap and usually includes some liquid, such as condensed stabilizer with some dissolved acetylene. The liquid may be coming from the walls of the inlet line 504 or in the form of the mist, i.e., small droplets suspended in the acetylene gas stream. The trap 500 may include a mist barrier 506 that assists in separating the liquid from the acetylene gas stream. The mist barrier 506 could be made of any suitable material that is resistant to stabilizer and acetylene and can withstand temperatures (up to −80° C.). In the certain embodiments, the mist barrier 506 is highly porous aluminum block (i.e. aluminum foam). The mist barrier 506 may have tortuous paths for the acetylene gas stream to pass through while trapping the liquid on the side of these paths. The liquid then flows back to the bottom of the trap 500. The porosity of the mist barrier 506 should be sufficiently open for the liquid to flow and not block the acetylene gas stream inside the trap. The mist trap 506 may also provide additional condensation surface for the stabilizer remaining in the acetylene gas stream.

As indicated, the condensed liquid gravitationally flows to the bottom of the trap 500. The liquid is then removed through the condensed liquid outlet 512. As explained with reference to FIG. 5, the removal of liquid may depend on a liquid level 510 at the bottom of the trap. When the liquid level 510 reaches a certain maximum value 510A, the draining valve of the pre-processing module is opened and the condensed liquid is drained through the condensed liquid outlet 512. Draining is then stopped when the liquid level 510 reaches or falls below a certain minimum value 510B. Monitoring of the liquid level 510 within the trap 500 is done through a sensor path 508 that provides a direct exposure of the liquid level to a liquid level sensor.

The acetylene gas stream then leaves the trap 500 through the gas line outlet 514. As explained above, the stream may then enter another heat exchanger where addition condensation may occur. Any additional condensation the outlet gas line 514 returns to the bottom of the trap 500.

### Deposition Chamber

The disclosed carbon deposition processes may be implemented in a plasma enhanced chemical vapor deposition (PECVD) reactor. Such a reactor may take many different forms. In certain embodiments, the apparatus will include one or more chambers or “reactors” (sometimes including multiple stations) that house one or more wafers and are suitable for wafer processing. During deposition, a chamber may hold one or more wafers for processing. The one or more chambers maintain the wafer in a defined position or positions during deposition. In one embodiment, a wafer undergoing hard mask deposition is transferred from one station to another within a reactor chamber during the process. For example, for certain hard mask film deposition processes, one-quarter of film thickness may be deposited at each of four stations in accordance with the disclosed embodiments. Of course, the full film deposition may occur entirely at a single station or any fraction of the total film thickness may be deposited at any number of stations.

While in process, each wafer is held in place by a pedestal, wafer chuck and/or other wafer holding apparatus. For certain operations in which the wafer is to be heated, the apparatus may include a heater such as a heating plate. In certain embodiments, a Vector® reactor, manufactured by Lam Research, Inc. of Fremont, Calif., may be used to implement the disclosed embodiments.

FIG. 6 provides a block diagram depicting various reactor components, which may be put into play to deposit a carbon-containing film from acetylene and in which the components can be controlled by way of a system controller 628. As shown, a reactor 600 includes a process chamber 624, which encloses other components of the reactor and serves to contain the plasma generated by a capacitor type system including a showerhead 614 working in conjunction with a grounded heater block 620. A high-frequency RF generator 604, connected to a matching network 606, and a low-frequency RF generator 602 are connected to showerhead 614. Alternatively, a low-frequency RF generator 602 may connected to the substrate 616. The power and frequency supplied by the matching network 606 is sufficient to generate a plasma from the process gas, for example 400-700 W total energy. In a typical process, the high frequency RF component is generally between 2-60 MHz; in certain embodiments, the HF component is about 13.56 MHz. The LF component can be from about 100 kHz-2 MHz; in certain embodiments, the LF component is 400 kHz

Within the reactor, a wafer pedestal 618 supports a substrate 616. The pedestal may include a chuck, a fork, or lift pins to hold and transfer the substrate during and between the deposition and/or plasma treatment reactions. The chuck may be an electrostatic chuck, a mechanical chuck or various other types of chuck as are available for use in the industry and/or research.

Process gases are introduced via an inlet 612. Multiple source gas lines 610 are connected to manifold 608. The gases may be premixed or not. Valving and mass flow control mechanisms may be employed to ensure that the correct gases are delivered during the deposition and plasma treatment phases of the process. In case the chemical precursor(s) is delivered in the liquid form, liquid flow control mechanisms are employed. The liquid is vaporized and mixed with other process gases prior to deposition.

Process gases exit chamber 600 via an outlet 622. A vacuum pump 626 (e.g., a one or two stage mechanical dry pump and/or a turbomolecular pump) typically draws process gases out and maintains a suitably low pressure within the reactor by a close loop-controlled flow restriction device, such as a throttle valve or a pendulum valve.

In some embodiments, there may be a user interface associated with a system controller 628. The user interface may include a display screen, graphical software displays of the apparatus and/or process conditions, and user input devices such as pointing devices, keyboards, touch screens, microphones, etc.

In some embodiments, parameters adjusted by the system controller 628 may relate to process conditions. Non-limiting examples include process gas composition and flow rates, temperature, pressure, plasma conditions (such as RF bias power levels), etc. These parameters may be provided to the user in the form of a recipe, which may be entered utilizing the user interface.

Signals for monitoring the process may be provided by analog and/or digital input connections of system controller 628 from various process tool sensors. The signals for controlling the process may be output on the analog and digital output connections of process tool 600. Non-limiting examples of process tool sensors that may be monitored include mass flow controllers, pressure sensors (such as manometers), thermocouples, etc. Appropriately programmed feedback and control algorithms may be used with data from these sensors to maintain process conditions.

The system controller 628 may provide program instructions for implementing the above-described deposition processes. The program instructions may control a variety of process parameters, such as DC power level, RF bias power level, pressure, temperature, etc. The instructions may control the parameters to operate dry development and/or etch processes according to various embodiments described herein.

The system controller 628 will typically include one or more memory devices and one or more processors configured to execute the instructions so that the apparatus will perform a method in accordance with disclosed embodiments. Machine-readable media containing instructions for controlling process operations in accordance with disclosed embodiments may be coupled to the system controller 628.

In some implementations, a controller is part of a system, which may be part of the above-described examples. Such systems can comprise semiconductor processing equipment, including a processing tool or tools, chamber or chambers, a platform or platforms for processing, and/or specific processing components (a wafer pedestal, a gas flow system, etc.). These systems may be integrated with electronics for controlling their operation before, during, and after processing of a semiconductor wafer or substrate. The electronics may be referred to as the “controller,” which may control various components or subparts of the system or systems. The controller, depending on the processing requirements and/or the type of system, may be programmed to control any of the processes disclosed herein, including the delivery of processing gases, temperature settings (e.g., heating and/or cooling), pressure settings, vacuum settings, power settings, radio frequency (RF) generator settings, RF matching circuit settings, frequency settings, flow rate settings, fluid delivery settings, positional and operation settings, wafer transfers into and out of a tool and other transfer tools and/or load locks connected to or interfaced with a specific system.

Broadly speaking, the controller may be defined as electronics having various integrated circuits, logic, memory, and/or software that receive instructions, issue instructions, control operation, enable cleaning operations, enable endpoint measurements, and the like. The integrated circuits may include chips in the form of firmware that store program instructions, digital signal processors (DSPs), chips defined as application specific integrated circuits (ASICs), and/or one or more microprocessors, or microcontrollers that execute program instructions (e.g., software). Program instructions may be instructions communicated to the controller in the form of various individual settings (or program files), defining operational parameters for carrying out a particular process on or for a semiconductor wafer or to a system. The operational parameters may, in some embodiments, be part of a recipe defined by process engineers to accomplish one or more processing steps during the fabrication of one or more layers, materials, metals, oxides, silicon, silicon dioxide, surfaces, circuits, and/or dies of a wafer.

The system controller 628, in some implementations, may be a part of or coupled to a computer that is integrated with, coupled to the system, otherwise networked to the system, or a combination thereof. For example, the system controller 628 may be in the “cloud” or all or a part of a fab host computer system, which can allow for remote access of the wafer processing. The computer may enable remote access to the system to monitor current progress of fabrication operations, examine a history of past fabrication operations, examine trends or performance metrics from a plurality of fabrication operations, to change parameters of current processing, to set processing steps to follow a current processing, or to start a new process. In some examples, a remote computer (e.g., a server) can provide process recipes to a system over a network, which may include a local network or the Internet. The remote computer may include a user interface that enables entry or programming of parameters and/or settings, which are then communicated to the system from the remote computer. In some examples, the system controller 628 receives instructions in the form of data, which specify parameters for each of the processing steps to be performed during one or more operations. It should be understood that the parameters may be specific to the type of process to be performed and the type of tool that the system controller 628 is configured to interface with or control. Thus, as described above, the system controller 628 may be distributed, such as by comprising one or more discrete controllers that are networked together and working towards a common purpose, such as the processes and controls described herein. An example of a distributed controller for such purposes would be one or more integrated circuits on a chamber in communication with one or more integrated circuits located remotely (such as at the platform level or as part of a remote computer) that combine to control a process on the chamber.

Without limitation, example systems may include a plasma etch chamber or module, a deposition chamber or module, a spin-rinse chamber or module, a metal plating chamber or module, a clean chamber or module, a bevel edge etch chamber or module, a physical vapor deposition (PVD) chamber or module, a chemical vapor deposition (CVD) chamber or module, an atomic layer deposition (ALD) chamber or module, an atomic layer etch (ALE) chamber or module, an ion implantation chamber or module, a track chamber or module, and any other semiconductor processing systems that may be associated or used in the fabrication and/or manufacturing of semiconductor wafers.

As noted above, depending on the process step or steps to be performed by the tool, the system controller 628 might communicate with one or more of other tool circuits or modules, other tool components, cluster tools, other tool interfaces, adjacent tools, neighboring tools, tools located throughout a factory, a main computer, another controller, or tools used in material transport that bring containers of wafers to and from tool locations and/or load ports in a semiconductor manufacturing factory.

As indicated, the disclosed embodiments may be implemented on a multi-station or a single station tool. In one embodiment, the pre-processing module is used for treating acetylene gas stream supplied to a semiconductor deposition chamber. Any deposition chamber that may use acetylene for semiconductor manufacturing can be used with pre-processing modules disclosed herein.

### Deposition Operating Conditions

Many of the process parameters listed here are appropriate for depositing carbon films using a Lam Research Vector® module having four stations for depositing an ashable hard mask on a 300 mm wafer. However, the disclosed embodiments apply more broadly to any semiconductor deposition chamber using acetylene gas stream for semiconductor processing. One skilled in the art will readily appreciate that the process parameters may be scaled based on the deposition chamber volume, wafer size, and other factors. For example, power outputs of low frequency (LF) and high frequency (HF) generators are typically directly proportional to the deposition surface area of the wafer. Similarly, flow rates depend on the free volume of the deposition chamber, which is 195 L for each of four deposition chambers in a Vector® deposition chamber.

Plasma may be generated using dual-frequency plasma generation process. For example, a low frequency (LF) generator may provide about 200-1000 W at about 50-400 kHz, while a high frequency (HF) generator may provide about 500-2,000 W at about 2-60 MHz during the deposition process. The deposition process may be performed when substrate temperature is between about 100° C. and 500° C. The pressure of the deposition chamber may be maintained at about 2-15 Torr. One example of process conditions for ashable hard mask deposition is summarized in Table 2. Deposition is continued until the desired thickness of film is deposited. According to various embodiments, between about 1,000 and 9,000 angstroms is deposited.

TABLE 2

| Parameter | Typical Process Range | |
|---|---|---|
| C₂H₂ Flow Rate | 1,000-10,000 | sccm |
| N₂ Flow Rate | 0-5,000 | sccm |
| He Flow Rate | 0-3,000 | sccm |
| H₂ Flow Rate | 0-10,000 | sccm |
| LF Power at Frequency | 0-2,400 W at 50-400 kHz | |
| HF Power at Frequency | 500-2,000 W at 2-60 MHz | |
| Pressure | 0.5-15 | Torr |
| Temperature | 150-700° | C. |
| Total Deposited Thickness | 250-10.000 | Å |

It should be understood that the disclosed embodiments are not limited to the deposition of ashable hardmask films at the process conditions above but apply to deposition of any carbon-based film using acetylene as a precursor in semiconductor processing. This includes, but is not limited to, plasma-enhanced CVD processes, thermal CVD processes, high density plasma CVD, atomic layer deposition (ALD) processes, etc. All of the above process conditions may be varied outside the example ranges shown in Table 2, so long as acetylene is used as a process gas.

While examples of flow rates are described above in Table 2, in certain embodiments, the methods disclosed herein are used with low flow rate processes, e.g., 100-1000 sccm acetylene flow or lower. Dilution at these low flow rates may be particularly detrimental to the repeatability, so the use of low vapor pressure stabilizers is advantageous.

Further, depending on deposition chamber size and other process parameters, the flow rate of acetylene may be about 3,000-10,000 sccm during the deposition process. In one embodiment, the flow rate of acetylene may be about 5,000-8,000 sccm. Other stages of semiconductor processing, such as cleaning of the chamber, may not involve carbon containing precursors. The process gas may also include other carbon containing precursors, such as methane, ethylene, propylene, butane, cyclohexane, benzene and toluene, and others.

A carrier gas may be used to dilute the precursor. The carrier gas may include any suitable carrier gas employed in semiconductor processing, such as helium, argon, nitrogen, hydrogen, or a combination of these. The overall carrier gas flow rate may depend on deposition chamber size and other process parameters and may range from about 500-10,000 sccm. In a specific embodiment nitrogen and helium are used as carrier gases having corresponding flow rates ranges of about 500-5,000 sccm and about 300-3,000 sccm. Other stages of semiconductor processing may include different processing gases and different flow rates.

In certain embodiments, a 300 mm Lam Research Vector® tool with four deposition stations is used to deposit an ashable hardmask onto a 300 mm wafer. In certain embodiments, the process includes four operations: undercoat deposition, ashable hard mask pre-coat, ashable hard mask deposition, and chamber cleaning. The acetylene gas stream may be used in the ashable hard mask pre-coat and/or ashable hard mask deposition operations. In certain embodiments, the acetylene gas stream that passes through the pre-processing module is delivered at a flow rate of about 7000 sccm during these operations. The dual-frequency PECVD module may provide Low Frequency (LF) power of about 200-600 W and High Frequency (HF) power at about 900-1500 W during these operations, while the process chamber was maintained at approximately 4-12 Torr.

### CONCLUSION

Although the foregoing embodiments have been described in some detail for purposes of clarity of understanding, it will be apparent that certain changes and modifications may be practiced within the scope of the appended claims. Embodiments disclosed herein may be practiced without some or all of these specific details. In other instances, well-known process operations have not been described in detail to not unnecessarily obscure the disclosed embodiments. Further, while the disclosed embodiments will be described in conjunction with specific embodiments, it will be understood that the specific embodiments are not intended to limit the disclosed embodiments. It should be noted that there are many alternative ways of implementing the processes, systems, and apparatus of the present embodiments. Accordingly, the present embodiments are to be considered as illustrative and not restrictive, and the embodiments are not to be limited to the details given herein.

1. A composition comprising a mixture or solution of:
acetylene; and
a stabilizer comprising an amide having a vapor pressure of about 3 Torr or lower at 25° C., an amine, an imine, a nitrile having a vapor pressure of about 80 Torr or lower at 25° C., a nitrogen-containing saturated heterocyclic ring compound, a nitrogenwavs1-containing unsaturated heterocyclic ring compound, an unsaturated linear or branched hydrocarbon, an unsaturated ring hydrocarbon having a vapor pressure of about 5 Torr or lower at 25° C., a non-aromatic unsaturated ring hydrocarbon, an ether, a ketone, an aldehyde, an ester having a vapor pressure of about 90 Torr or lower at 25° C., an ionic liquid, a carbene, a silylene, a mixed electron donor compound comprising a pi bond and an atom having a lone electron pair, a phosphorus-containing compound in which the phosphorus atom has a lone electron pair, or a sulfur-containing compound in which the sulfur atom has a lone electron pair,
wherein the ketone has a vapor pressure of about 30 Torr or lower at 25° C.; or wherein the ketone is selected from the group consisting of acetylacetone (acac), 2-butanone, 2-pentanone, 2-hexanone, 2-heptanone, 2-octanone, 2-nonanone, 2-decanone, 3-pentanone, 3-heptanone, 3-octanone, 3-nonanone, 3-decanone, and aromatic ketones comprising acetophenone, 3-hydroxyacetophenone, cyclohexanone, benzophenone, butyrophenone, acetylpyrazine, 2-acetyl pyridine, acrylophenone, capillin, dibenzoylmethane, indenone, 1-indanone, paroxypropione, phenylglyoxal, piceol, propriophenone, pyridoxal, 2,4,6-trihydroxyacetophenone, 2,4,5-trihydroxyacetophenone, or valerophenone.

2. The composition of claim 1, wherein the stabilizer is the ionic liquid comprising a cation selected from the group consisting of imidazolium, pyridinium, ammonium, phosphonium, thiazolium, and triazolium.

3. The composition of claim 1, wherein the stabilizer is the carbene selected from the group consisting of transition metal carbene complexes, N-heterocyclic carbenes, and methylenes.

4. The composition of claim 1, wherein the stabilizer is the silylene or an N-heterocyclic silylene.

5. The composition of claim 1, wherein the stabilizer comprises the ketone having a vapor pressure of about 30 Torr or lower at 25° C.

6. The composition of claim 1, wherein the stabilizer comprises the ketone selected from the group consisting of acetylacetone (acac), 2-butanone, 2-pentanone, 2-hexanone, 2-heptanone, 2-octanone, 2-nonanone, 2-decanone, 3-pentanone, 3-heptanone, 3-octanone, 3-nonanone, 3-decanone, and aromatic ketones comprising acetophenone, 3-hydroxyacetophenone, cyclohexanone, benzophenone, butyrophenone, acetylpyrazine, 2-acetyl pyridine, acrylophenone, capillin, dibenzoylmethane, indenone, 1-indanone, paroxypropione, phenylglyoxal, piceol, propriophenone, pyridoxal, 2,4,6-trihydroxyacetophenone, 2,4,5-trihydroxyacetophenone, or valerophenone.

7. The composition of claim 1, wherein the stabilizer comprises the aldehyde.

8. (canceled)

9. The composition of claim 1, wherein the stabilizer comprises the ester having a vapor pressure of about 90 Torr or lower at 25° C.

10. The composition of claim 1, wherein the stabilizer comprises the amide having a vapor pressure of about 3 Torr or lower at 25° C.

11. The composition of claim 1, wherein the stabilizer comprises the ether.

12. (canceled)

13. The composition of claim 1, wherein the stabilizer comprises the amine.

14-16. (canceled)

17. The composition of claim 1, wherein the stabilizer comprises the imine.

18. (canceled)

19. The composition of claim 1, wherein the stabilizer comprises the nitrile having a vapor pressure of about 80 Torr or lower at 25° C.

20. The composition of claim 1, wherein the stabilizer comprises the nitrogen-containing saturated heterocyclic ring compound.

21. (canceled)

22. The composition of claim 1, wherein the stabilizer comprises the nitrogen-containing unsaturated heterocyclic ring compound.

23. (canceled)

24. The composition of claim 1, wherein the stabilizer comprises the mixed electron donor compound comprising a pi bond and an atom having a lone electron pair.

25. (canceled)

26. The composition of claim 1, wherein the stabilizer comprises the phosphorus-containing compound in which the phosphorus atom has a lone electron pair.

27. (canceled)

28. The composition of claim 1, wherein the stabilizer comprises the sulfur-containing compound in which the sulfur atom has a lone electron pair.

29. (canceled)

30. The composition of claim 1, wherein the stabilizer comprises the unsaturated linear or branched hydrocarbon.

31. (canceled)

32. The composition of claim 1, wherein the stabilizer comprises the unsaturated ring hydrocarbon having a vapor pressure of about 5 Torr or lower at 25° C.

33. The composition of claim 1, wherein the stabilizer comprises the non-aromatic unsaturated ring hydrocarbon.

34-35. (canceled)

36. The composition of claim 1, wherein the acetylene and stabilizer are stored under a pressure of at least about 200 psi.

37. A stabilized composition comprising:
a pressurized acetylene; and
a stabilizer is a stabilizer comprising a nitrogen atom, wherein the stabilizer is not dimethylformamide, not dimethylacetamide, not N-methyl-2-pyrrolidone, and not acetonitrile; a heterocycle, wherein the stabilizer is not 1,3-dioxolane, not 1,4-dioxane, and not N-methyl-2-pyrrolidone; a substituted aromatic hydrocarbon having one or more substitutions, wherein the one or more substitutions are selected from the group consisting of halo, amine, and optionally substituted C₂₋₈ alkyl; an optionally substituted alkene or an optionally substituted alkyne; an aldehyde or an ether, wherein the stabilizer is not 1,3-dioxolane and not 1,4-dioxane; an ester selected from the group consisting of a cyclic ester, a glycol based ester, a lactate, a carbonate ester, an amino ester, and a diester; a cyclic ketone, an aryl ketone, a dione, or a trione; a carbene or a carbene derivative; a metal compound, an onium compound, an organosulfur compound, or an organophosphorus compound, wherein the stabilizer is not dimethylsulfoxide; or an ionic liquid.

38. The stabilized composition of claim 37, wherein the stabilizer is the stabilizer comprising the nitrogen atom and further comprises optionally substituted heterocyclyl.

39. The stabilized composition of claim 37, wherein the stabilizer is the stabilizer comprising the nitrogen atom and is an amide, an amine, a guanidine, an imine, or an N-heterocyclic carbene.

40. The stabilized composition of claim 37, wherein the stabilizer is the stabilizer comprising the nitrogen atom and is an amide selected from the group consisting of a dialkyl amide, a pyrrolidone, an acetamide, a morpholide, an ester amide, and a cyclic amide.

41. The stabilized composition of claim 37, wherein the stabilizer is the stabilizer comprising the nitrogen atom and is an amine comprising optionally substituted aliphatic, optionally substituted alkyl, optionally substituted aryl, optionally substituted aliphatic-aryl, optionally substituted alkyl-aryl, optionally substituted alkenyl-aryl, optionally substituted alkynyl-aryl, optionally substituted heteroaliphatic, optionally substituted heteroalkyl, optionally substituted heterocyclyl, or optionally substituted alkyl-heterocyclyl.

42. The stabilized composition of claim 37, wherein the stabilizer is the stabilizer comprising the nitrogen atom and is a Schiff base.

43. The stabilized composition of claim 37, wherein the stabilizer is the heterocycle, and wherein the stabilizer is not 1,3-dioxolane, not 1,4-dioxane, and not N-methyl-2-pyrrolidone.

44-45. (canceled)

46. The stabilized composition of claim 37, wherein the stabilizer is the substituted aromatic hydrocarbon having one or more substitutions, and wherein the one or more substitutions are selected from the group consisting of halo, amine, and optionally substituted C₂₋₈ alkyl.

47. (canceled)

48. The stabilized composition of claim 37, wherein the stabilizer is the optionally substituted alkene or the optionally substituted alkyne.

49. (canceled)

50. The stabilized composition of claim 37, wherein stabilizer is the aldehyde or the ether, and wherein the stabilizer is not 1,3-dioxolane and not 1,4-dioxane.

51-52. (canceled)

53. The stabilized composition of claim 37, wherein the stabilizer is the ester selected from the group consisting of a cyclic ester, a glycol based ester, a lactate, a carbonate ester, an amino ester, and a diester.

54. (canceled)

55. The stabilized composition of claim 37, wherein the stabilizer is the cyclic ketone, the aryl ketone, the dione, or the trione.

56. (canceled)

57. The stabilized composition of claim 37, wherein the stabilizer is the carbene or the carbene derivative.

58-60. (canceled)

61. The stabilized composition of claim 37, wherein the stabilizer is the metal compound, the onium compound, the organosulfur compound, or the organophosphorus compound, wherein the stabilizer is not dimethylsulfoxide.

62-64. (canceled)

65. The stabilized composition of claim 37, wherein the stabilizer is the ionic liquid.

66. (canceled)
